SEQUENCE LISTING (11 sequences)

SEQ ID NO: 1 - protein, 5 aa, Artificial Sequence
  Synthetic Construct
DYWMQ

SEQ ID NO: 2 - protein, 17 aa, Artificial Sequence
  Synthetic Construct
TIYPGDGDTGYAQKFQG

SEQ ID NO: 3 - protein, 11 aa, Artificial Sequence
  Synthetic Construct
GDYYGSNSLDY

SEQ ID NO: 4 - protein, 11 aa, Artificial Sequence
  Synthetic Construct
KASQDVSTVVA

SEQ ID NO: 5 - protein, 7 aa, Artificial Sequence
  Synthetic Construct
SASYRYI

SEQ ID NO: 6 - protein, 9 aa, Artificial Sequence
  Synthetic Construct
QQHYSPPYT

SEQ ID NO: 7 - protein, 120 aa, Artificial Sequence [sequence omitted]

SEQ ID NO: 8 - protein, 108 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 9 - protein, 108 aa, Artificial Sequence
  Synthetic Construct
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 10 - protein, 449 aa, Artificial Sequence
  Synthetic Construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0135696 A1
(43) Pub. Date: May 5, 2022

(54) USE OF ISATUXIMAB FOR THE TREATMENT OF MULTIPLE MYELOMA

(71) Applicant: Sanofi-Aventis U.S. LLC, Bridgewater, NJ (US)

(72) Inventors: Dorothee SEMIOND, Paris (FR); Hoai-Thu THAI, Paris (FR); Helgi VAN DE VELDE, Paris (FR); Christine VEYRAT-FOLLET, Paris (FR)

(21) Appl. No.: 17/517,567

(22) Filed: Nov. 2, 2021

Related U.S. Application Data
(60) Provisional application No. 63/239,108, filed on Aug. 31, 2021.
(60) Provisional application No. 63/109,305, filed on Nov. 3, 2020.

(51) Int. Cl. C07K 16/28; A61P 35/00; A61K 9/00

(52) U.S. Cl. CPC C07K 16/2896; A61K 2039/545; A61K 9/0019; A61P 35/00

(57) ABSTRACT

The present disclosure provides methods for treating multiple myeloma that comprise administering isatuximab to the individual at a dose of 10 mg/kg on Days 1, 8, 15, and 22 of a first 28-day cycle; administering the isatuximab at a dose of 10 mg/kg on Days 1 and 15 of a 28-day cycle, e.g., for at least 11 cycles, or e.g., until the individual achieves or is determined to achieve at least Very Good Partial Response (VGPR) to treatment; and administering the isatuximab at a dose of 10 mg/kg once every 28 days of one or more additional 28-day cycles.

### CROSS REFERENCE TO RELATED APPLICATIONS

This application claims priority to U.S. Provisional Application No. 63/109,305, filed Nov. 3, 2020 and U.S. Provisional Application No. 63/239,108, filed Aug. 31, 2021; each of which is incorporated herein by reference for all purposes

### FIELD

The present disclosure relates to methods of treating multiple myeloma by administering an anti-CD38 antibody, e.g., isatuximab.

### BACKGROUND

Multiple myeloma (MM) is a malignant plasma cell disease that is characterized by clonal proliferation of plasma cells in the bone marrow (BM) and the production of excessive amounts of a monoclonal immunoglobulin (usually of the IgG or IgA type or free urinary light chain, i.e., paraprotein, M-protein or M-component). Patients with MM can experience bone pain, bone fractures, fatigue, anemia, infections, hypercalcemia, and kidney problems (Rollig et al. (2015) Lancet. 385(9983):2197-208). The expression of CD38 is especially notable in MM as >98% of patients are positive for this protein (Goldmacher et al. (1994) Blood. 84(9):3017-25; Lin et al. (2004) Am J Clin Pathol. 121(4):482-8). The strong and uniform expression of CD38 on malignant clonal MM cells contrasts with the restricted expression pattern on normal cells suggesting this antigen may be useful for specific targeting of tumor cells.

In general, MM patients will receive treatment regimens during their lifespan that include such agents such as proteasome inhibitors (e.g., bortezomib, ixazomib, and carfilzomib) and immune modulatory agents or “IMiDs®” (e.g., lenalidomide, pomalidomide, and thalidomide), monoclonal antibodies (e.g., elotuzumab), histone deacetylase (HDAC) inhibitors (e.g., panobinostat) alone or in combination.

Determining the appropriate dosing schedule for antibodies is complicated by potential target-mediated drug disposition and tumor burden. The pharmacokinetics of a given antibody must be empirically determined. Whether and when a patient should receive antibody dosing at longer intervals (monthly versus every other week, for example) while preserving patient benefit must be separately evaluated for each antibody-based therapy.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some embodiments, provided is a method of treating a human individual having multiple myeloma, comprising: administering isatuximab to the individual at a dose of 10 mg/kg on Days 1, 8, 15, and 22 of a first 28-day cycle; administering the isatuximab at a dose of 10 mg/kg on Days 1 and 15 of a 28-day cycle for at least 11 cycles; and administering the isatuximab at a dose of 10 mg/kg once every 28 days of one or more additional 28-day cycles following the at least 11 cycles. In some embodiments, provided is a method of treating a human individual having multiple myeloma, comprising administering isatuximab to the individual at a weekly dose of 10 mg/kg of a first one-month cycle; administering the isatuximab at a dose of 10 mg/kg once every two weeks of a one-month cycle for at least 11 cycles following the first one-month cycle; and administering the isatuximab at a monthly dose of 10 mg/kg for one or more additional one-month cycles following the at least 11 cycles.

In some embodiments, provided is a method of treating a human individual having multiple myeloma, comprising: administering isatuximab to the individual at a dose of 10 mg/kg on Days 1, 8, 15, and 22 of a first 28-day cycle; administering the isatuximab at a dose of 10 mg/kg on Days 1 and 15 of one or more 28-day cycles after the first 28-day cycle until the individual achieves a response of at least very good partial response (VGPR); and administering the isatuximab at a dose of 10 mg/kg once every 28 days of one or more additional 28-day cycles after the individual achieves the response of at least VGPR. In some embodiments, provided is a method of treating a human individual having multiple myeloma, comprising: administering an anti-CD38 antibody to the individual at a weekly dose of 10 mg/kg of a first one-month cycle; administering the anti-CD38 antibody at a dose of 10 mg/kg once every two weeks of one or more one-month cycles after the first one-month cycle until the individual achieves a response of at least very good partial response (VGPR); and administering the anti-CD38 antibody at a dose of 10 mg/kg once a month for one or more additional one-month cycles after the individual achieves the response of at least VGPR.

In some embodiments, provided is a method of treating a human individual having multiple myeloma, comprising: administering the isatuximab to the individual at a dose of 10 mg/kg on Days 1, 8, 15, and 22 of first 28-day cycle; administering the isatuximab at a dose of 10 mg/kg on Days 1 and 15 of one or more 28-day cycles after the first 28-day cycle; measuring the individual's response to the treatment at one or more time points during the one or more 28-day cycles after the first 28-day cycle and selecting individuals who have at least a Very Good Partial Response (VGPR); and administering the isatuximab at a dose of 10 mg/kg once every 28 days of one or more additional 28-day cycles to the selected individuals. In some embodiments, provided is a method of treating human individuals having multiple myeloma, comprising: administering an anti-CD38 antibody to the individuals at weekly dose of 10 mg/kg of a first one-month cycle; administering the anti-CD38 antibody at a dose of 10 mg/kg on once every two weeks of one or more one-month cycles after the first one-month cycle; measuring the individuals' responses to the treatment at one or more time points during the one or more one-month cycles after the first one-month cycle and selecting the individuals who have at least a Very Good Partial Response (VGPR); and administering the anti-CD38 antibody at a dose of 10 mg/kg once a month for one or more additional one-month cycles to the selected individuals.

In some embodiments, provided is a method of treating a human individual having multiple myeloma, comprising: measuring the individual's serum and urine M-protein at a first time point prior to administration of isatuximab; administering the isatuximab to the individual at a dose of 10 mg/kg on Days 1, 8, 15, and 22 of a first 28-day cycle; administering the isatuximab at a dose of 10 mg/kg on Days 1 and 15 of one or more 28-day cycles after the first 28-day cycle; measuring the individual's serum and/or urine M-protein at a second time point during the at least one or more 28-day cycles after the first 28-day cycle, and administering the isatuximab at a dose of 10 mg/kg once every 28 days of or more additional 28-day cycles if (a) the individual's serum M-protein level at the second time point is reduced by at least 90% as compared to the individual's serum M-protein level at the first time point and (b) the individual's urine M-protein level at the second time point is less than 100 mg/24 hours. In some embodiments, provided is a method of treating a human individual having multiple myeloma, comprising: measuring the individual's serum and urine M-protein at a first time point prior to administration of an anti-CD38 antibody; administering the anti-CD38 antibody to the individual at a weekly dose of 10 mg/kg of a first one-month cycle; administering the anti-CD38 antibody at a dose of 10 mg/kg once every two weeks of one or more one-month cycles after the first one-month cycle; measuring the individual's serum and/or urine M-protein at a second time point during the at least one or more one-month cycles after the first one-month cycle, and administering the anti-CD38 antibody at a monthly dose of 10 mg/kg for one or more additional one-month cycles if (a) the individual's serum M-protein level at the second time point is reduced by at least 90% as compared to the individual's serum M-protein level at the first time point and (b) the individual's urine M-protein level at the second time point is less than 100 mg/24 hours.

In some embodiments, provided is a method of treating a human individual having multiple myeloma, comprising: measuring the individual's serum and/or urine M-protein level prior to administration of isatuximab; administering the isatuximab to the individual at a dose of 10 mg/kg on Days 1, 8, 15, and 22 of a first 28-day cycle; administering the isatuximab at a dose of 10 mg/kg on Days 1 and 15 of one or more 28 day cycles after the first 28-day cycle until (a) the individual's serum M-protein level at reduced by at least 90% as compared to the serum M-protein level prior to the administration of isatuximab and (b) the individual's urine M-protein level is less than 100 mg/24 hours; and administering the isatuximab at a dose of 10 mg/kg once every 28 days of one or more additional 28-day cycles after (a) the individual's serum M-protein level is determined to be reduced by at least 90% as compared to the individual's serum M-protein level at the first time point and (b) the individual's urine M-protein level is determined to be less than 100 mg/24 hours. In some embodiments, (a) the reduction in the individual's serum M-protein level and (b) the individual's urine M-protein level of less than 100 mg/24 hours are maintained for at least about any one of 6, 7, 8, 8, 9, 10, 11, or 12 months prior to administering the isatuximab at a dose of 10 mg/kg on Day 1 of every 28-day cycle. In some embodiments, provided is a method of treating a human individual having multiple myeloma, comprising: measuring the individual's serum and/or urine M-protein level prior to administration of anti-CD38 antibody; administering the anti-CD38 antibody to the individual at a weekly dose of 10 mg/kg of a first one-month cycle; administering the isatuximab at a dose of 10 mg/kg once every two weeks of one or more one-month cycles after the first one-month cycle until (a) the individual's serum M-protein level at reduced by at least 90% as compared to the serum M-protein level prior to the administration of the anti-CD38 antibody and (b) the individual's urine M-protein level is less than 100 mg/24 hours; and administering the anti-CD38 antibody at a dose of 10 mg/kg once a month for one or more additional one-month cycles after (a) the individual's serum M-protein level is determined to be reduced by at least 90% as compared to the individual's serum M-protein level at the first time point and (b) the individual's urine M-protein level is determined to be less than 100 mg/24 hours. In some embodiments, (a) the reduction in the individual's serum M-protein level and (b) the individual's urine M-protein level of less than 100 mg/24 hours are maintained for at least about any one of 6, 7, 8, 8, 9, 10, 11, or 12 months prior to administering the isatuximab at a dose of 10 mg/kg once a month for one or more additional one-month cycles.

In some embodiments, the individual's response to treatment is measured by assessing M-protein level in the blood and/or urine of the individual. In some embodiments, the M-protein level in the individual's blood and/or urine is assessed via immunofixation and/or electrophoresis. In some embodiments, the response of at least VGPR is maintained for at least about 6 months prior to administering the isatuximab once every 28 days, or once every month, of one or more 28-day cycles. In some embodiments, the response of at least VGPR is maintained for at least about 12 months prior to administering the isatuximab once every 28 days, or once every month, of one or more 28-day cycles. In some embodiments, the isatuximab is administered at a dose of 10 mg/kg on Days 1 and 15 of one or more 28 day cycles, or once every two weeks of the one or more one-month cycles, for at least 11 cycles prior to administering the isatuximab once every 28 days of one or more 28-day cycles, or once every two weeks of the one or more one-month cycles. In some embodiments, the isatuximab is administered at a dose of 10 mg/kg on Days 1 and 15 of one or more 28 day cycles, or once every two weeks of the one or more one-month cycles, for at least 23 cycles prior to administering the isatuximab once every 28 days of one or more 28-day cycles, or once every two weeks of the one or more one-month cycles. In some embodiments, the treatment extends the progression free survival (PFS) of the individual.

In some embodiments, the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody comprises a heavy chain variable region (VH) comprising an amino acid sequence of SEQ ID NO: 7 and a light chain variable region (VL) comprising an amino acid sequence of SEQ ID NO: 8 or SEQ ID NO: 9. In some embodiments, the anti-CD38 antibody is isatuximab.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a schematic representation of the integrated drug disease model that integrates kinetic-pharmacodynamic models (K-PD) for pomalidomide (Pom), dexamethasone (Dex) and the pharmacokinetic (PK) model for isatuximab, tumor growth inhibition, and progression free survival (PFS).

FIG. 2 shows individual fits of serum M-protein time course and PFS probability in 6 illustrative patients, 3 with the observed event and 3 with censored event. Patients in the Isa-Pd arm are in the middle and on the left; patients in the Pd arm are on the right. Blue dots denote the serum M-protein observations and red dots BLQ observations. The green curves denote the longitudinal predictions using the joint model. The vertical lines show the status of the patients (solid: progression event occurred, dashed: censored). The red solid curves denote the PFS probability predicted by the joint model. The black curves represent the predicted value of the current slope of serum M-protein kinetics. BLQ, below the limit of quantification; Isa, isatuximab; MP, M-protein; Pd, pomalidomide and dexamethasone; PFS, progression-free survival.

FIG. 3 shows visual predictive checks for the PFS and longitudinal part of the final joint model. The shaded area and the dotted lines represent the 90% prediction interval and the predicted median of 5th, 50th and 95th percentiles of simulated data (n=1000). The solid lines represent the 5th, 50th and 95th percentiles of observed longitudinal data or the observed Kaplan-Meier estimate (with its 90th confidence interval in thin dashed black lines). CI, confidence interval; I, isatuximab; KM, Kaplan Meier; M-P, M-protein; Pd, pomalidomide and dexamethasone; PFS, progression-free survival; PI, prediction interval.

FIG. 4 shows the impact of covariate effects on serum M-protein kinetics and PFS probability of not progressed; total serum M-protein population (n=256). ALBN, albumin; B2MG, β2-microglobulin; Ig, immunoglobulin; PFS, progression-free survival.

FIGS. 5A-5G provide model evaluations of the best joint serum M-protein and PFS model. (ALBN, albumin; B2MG, β2-microglobulin; PFS, progression-free survival; IG, immunoglobulin; I, isatuximab; LOQ, limit of quantification; M-Prot, M-protein; PCYTOMA, presence of plasmacytomas; Pd, pomalidomide/dexamethasone; PFS, progression-free survival; VPC, visual predictive check). FIG. 5A provides observations vs individual predictions of serum M-protein. FIG. 5B provides individual weighted residuals (IWRES) vs time (days) or vs individual prediction for serum M-protein (g/L). FIG. 5C provides prediction corrected (PC) VPC for the longitudinal part, stratified by arm. FIG. 5D shows predicted individual PFS probability. FIG. 5E shows Cox-Snell residuals. FIG. 5F shows deviance residuals stratified by covariates. FIG. 5G shows de-trended prediction distribution (pd) for time to event (TTE) data over time and stratified by arm.

FIG. 6 shows characteristics of patients with no risk of earlier progression with the new, hypothetical dosing regimen compared to the standard one (n=60) versus others (n=44). ALBN, albumin; B2MG, β2-microglobulin; BMPC, bone marrow plasma cells; GFR, glomerular filtration rate; W24, week 24.

FIG. 7 shows a posterior predictive check of PFS HR using the joint model. Green zone: 95% prediction interval, black bar: predicted median HR, red bar: observed HR. PFS, progression-free survival; HR, hazard ratio.

### DETAILED DESCRIPTION

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

“Sustained response” refers to the sustained effect on preventing or delaying progression of a disease (e.g., multiple myeloma) and/or improving one or more response criteria after cessation of a treatment. For example, response to treatment for multiple myeloma may be measured according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473. (See also Table A below.) In some embodiments, the sustained response has a duration at least the same as the treatment duration, at least 1.5×, 2.0×, 2.5×, or 3.0× length of the treatment duration.

TABLE A
Standard International Myeloma Working Group (IMWG) Response Criteria

| Response | IMWG criteria |
|---|---|
| Complete | Negative immunofixation on the serum and urine and |
| Response (CR) | disappearance of any soft tissue plasmacytomas and < 5% |
| | plasma cells in bone marrow aspirates. |
| | Two consecutive assessments are needed. No known evidence of progressive |
| | disease or new bone lesions if radiographic studies were performed |
| Stringent | CR as defined above plus: |
| Complete | Normal free light chain ratio (0.26 to 1.65) and |
| Response | Absence of clonal cells in bone marrow by immunohistochemistry |
| (sCR) | (κ/λ ratio ≤ 4:1 or ≥ 1:2 for κ and λ patients, respectively, after |
| | counting ≥ 100 plasma cells). |
| | Two consecutive assessments of laboratory parameters are needed. No |
| | known evidence of progressive disease or new bone lesions if radiographic |
| | studies were performed |
| Very Good | Serum and urine M protein detectable by immunofixation but not on |
| Partial | electrophoresis or ≥ 90% reduction in serum M protein plus urine M |
| Response | protein level < 100 mg/24 hour. ≥90% decrease in the sum of the products |
| (VGPR) | of maximal perpendicular diameter (SPD) compared to baseline in |
| | soft tissue plasmacytoma. |
| | Two consecutive assessments are needed. No known evidence of progressive |
| | disease or new bone lesions if radiographic studies were performed. |
| Partial | ≥50% reduction of serum M protein and reduction in 24 hours urinary M |
| Response (PR) | protein by ≥ 90% or to < 200 mg/24 hour |
| | In addition to the above listed criteria, if present at baseline, a ≥ 50% reduction |
| | in the size (sum of the products of the maximal perpendicular diameters or |
| | “SPD”) of soft tissue plasmacytomas is also required |
| | Two consecutive assessments are needed. No known evidence of progressive |
| | disease or new bone lesions if radiographic studies were performed. |
| Minimal | ≥25% but ≤ 49% reduction in serum M protein and reduction in 24 h urine M |
| Response | protein by 50 to 89%, which still exceed 200 mg/24 hour. |
| (MR) | In addition to the above listed criteria, if present at baseline, ≥ 50% reduction |
| | in size (SPD) of soft tissue plasmacytomas is also required. |
| | Two consecutive assessments are needed. No known evidence of progressive |
| | disease or new bone lesions if radiographic studies were performed. |
| Stable Disease | Not meeting criteria for CR, VGPR, PR, MR or progressive disease. |
| (SD) | No known evidence of progressive disease or new bone lesions if |
| | radiographic studies were performed. |
| Progressive | Any 1 or more of the following criteria: Increase of ≥ 25% from lowest |
| disease | confirmed value in any 1 of the following criteria: |
| (PD) | Serum M protein (the absolute increase must be ≥ 0.5 g/dL). |
| | Serum M protein increase ≥ 1 g/dL if the lowest M component was ≥ 5 g/dL. |
| | Urine M-component (the absolute increase must be ≥ 200 mg/24 hour). |
| | Appearance of new lesion(s), ≥ 50% increase from nadir in SPD of > 1 lesion, |
| | or ≥ 50% increase in the longest diameter of a previous lesion > 1 cm in |
| | short axis. |
| | Two consecutive assessments are needed for PD on M protein. |

‡SPD, sum of the products of the maximal perpendicular diameters of measured lesions

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those that can reasonably be administered to a subject mammal to provide an effective dose of the active ingredient employed.

As used herein, the term “treatment” refers to clinical intervention designed to alter the natural course of the disease or cell (e.g., cancer cell) being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the individual/patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise, in order, the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs.

The term “Fc” as used herein refers to the sequence of a non-antigen-binding fragment that would result from digestion of an antibody or produced by other means, whether in monomeric or multimeric form, and can contain the hinge region. The original immunoglobulin source of the native Fc is preferably of human origin and can be any of the immunoglobulins. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, IgGA2, and IgG4). One example of a Fc is a disulfide-bonded dimer resulting from papain digestion of an IgG. The term “native Fc” as used herein is generic to the monomeric, dimeric, and multimeric forms.

As used herein, the term “overall response rate” or “ORR” refers to the proportion of individuals/patients with stringent complete response (sCR), complete response (CR), very good partial response (VGPR), and partial response (PR), as assessed by the IRC using the IMWG response criteria described in Kumar et al. (2016) “International Myeloma Working Group consensus criteria for response and minimal residual disease assessment in multiple myeloma.” Lancet Oncol. 17(8): e328-e346 and Durie et al. (2006) “International uniform response criteria for multiple myeloma. Leukemia. 20: 1467-1473. See also Table A herein.

### Overview

Provided herein are methods for treating or delaying the progression of multiple myeloma in an individual who has received one, two, three, or more than three prior therapies for multiple myeloma. The methods comprise administering to the individual an effective amount of an anti-CD38 antibody (e.g., isatuximab), carfilzomib, and dexamethasone. In some embodiments, the treatment extends the progression free survival (PFS) and/or the overall survival (OS) of the individual. In some embodiments, the treatment extends the progression free survival (PFS) and/or the overall survival (OS) of the individual, as compared to an individual who is not receiving treatment. In some embodiments, the treatment extends the progression free survival (PFS) and/or the overall survival (OS) of the individual, as compared to an individual receiving treatment with of carfilzomib and dexamethasone, but without the anti-CD38 antibody (e.g., isatuximab). In some embodiments, the individual is negative for minimal residual disease (MRD) (e.g., at a threshold of 10⁻⁴ or less, 10⁻⁵ or less, or 10⁻⁶ or less) after treatment.

### Anti-CD38 Antibodies

In some embodiments, the anti-CD38 antibody binds to human CD38. In some embodiments, the anti-CD38 antibody is a human antibody, a humanized antibody, or a chimeric antibody. In some embodiments, the anti-CD38 antibody comprises (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody comprises a heavy chain variable domain (V_{H}) that comprises an amino acid sequence that is at least 90% identical (e.g., at least any one of 91%, 92%, 94%, 95%, 96%, 97%, 98%, or 99%, including any range between these values) to SEQ ID NO: 7. Additionally or alternatively, in some embodiments, the anti-CD38 antibody comprises a light chain variable domain (V_{L}) that comprises an amino acid sequence that is at least 90% identical (e.g., at least any one of 91%, 92%, 94%, 95%, 96%, 97%, 98%, or 99%, including any range between these values) to SEQ ID NO: 8 or SEQ ID NO: 9. In some embodiments, the anti-CD38 antibody comprises a V_{H} that comprises SEQ ID NO: 7 and a V_{L} that comprises SEQ ID NO: 8 or SEQ ID NO: 9.

(SEQ ID NO: 7)
QVQLVQSGAE VAKPGTSVKL SCKASGYTFT
DYWMQWVKQR PGQGLEWIGT IYPGDGDTGY
AQKFQGKATL TADKSSKTVY MHLSSLASED
SAVYYCARGD YYGSNSLDYW GQGTSVTVSS
(SEQ ID NO: 8)
DIVMTQSHLS MSTSLGDPVS ITCKASQDVS
TVVAWYQQKP GQSPRRLIYS ASYRYIGVPD
RFTGSGAGTD FTFTISSVQA EDLAVYYCQQ
HYSPPYTFGG GTKLEIKR
(SEQ ID NO: 9)
DIVMAQSHLS MSTSLGDPVS ITCKASQDVS
TVVAWYQQKP GQSPRRLIYS ASYRYIGVPD
RFTGSGAGTD FTFTISSVQA EDLAVYYCQQ
HYSPPYTFGG GTKLEIKR

In some embodiments, the anti-CD38 antibody is isatuximab (CAS Registry Number: 1461640-62-9). Isatuximab, also known as hu38SB19 and SAR650984, is an anti-CD38 antibody described in WO 2008/047242 and U.S. Pat. No. 8,153,765, the contents of both of which are incorporated by reference herein in their entirety.

The heavy chain of isatuximab comprises the amino acid sequence:

(SEQ ID NO: 10)
QVQLVQSGAE VAKPGTSVKL SCKASGYTFT
DYWMQWVKQR PGQGLEWIGT IYPGDGDTGY
AQKFQGKATL TADKSSKTVY MHLSSLASED
SAVYYCARGD YYGSNSLDYW GQGTSVTVSS
ASTKGPSVFP LAPSSKSTSG GTAALGCLVK
DYFPEPVTVS WNSGALTSGV HTFPAVLQSS
GLYSLSSVVT VPSSSLGTQT YICNVNHKPS
NTKVDKKVEP KSCDKTHTCP PCPAPELLGG
PSVFLFPPKP KDTLMISRTP EVTCVVVDVS
HEDPEVKFNW YVDGVEVHNA KTKPREEQYN
STYRVVSVLT VLHQDWLNGK EYKCKVSNKA
LPAPIEKTIS KAKGQPREPQ VYTLPPSRDE
LTKNQVSLTC LVKGFYPSDI AVEWESNGQP
ENNYKTTPPV LDSDGSFFLY SKLTVDKSRW
QQGNVFSCSV MHEALHNHYT QKSLSLSPG

and the light chain of isatuximab comprises the amino acid sequence:

(SEQ ID NO: 11)
DIVMTQSHLS MSTSLGDPVS ITCKASQDVS
TVVAWYQQKP GQSPRRLIYS ASYRYIGVPD
RFTGSGAGTD FTFTISSVQA EDLAVYYCQQ
HYSPPYTFGG GTKLEIKRTV AAPSVFIFPP
SDEQLKSGTA SVVCLLNNFY PREAKVQWKV
DNALQSGNSQ ESVTEQDSKD STYSLSSTLT
LSKADYEKHK VYACEVTHQG LSSPVTKSFN
RGEC

The anti-CD38 antibodies may be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. The vector is typically transformed into a host cell suitable for expression of the nucleic acid. In some embodiments, the host cell is a eukaryotic cell or a prokaryotic cell. In some embodiments, the eukaryotic host cell is a mammalian cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MOCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268. The anti-CD38 antibody prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In general, various methodologies for preparing antibodies for use in research, testing, and clinical applications are well-established in the art, consistent with the above-described methodologies and/or as deemed appropriate by one skilled in the art.

### Pharmaceutical Compositions and Formulations

Also provided herein are pharmaceutical compositions and formulations, e.g., for the treatment of multiple myeloma (such as refractory multiple myeloma or relapsed and refractory multiple myeloma) comprising an anti-CD38 antibody (such as isatuximab), carfilzomib, or dexamethasone. In some embodiments, each of the anti-CD38 antibody (e.g., isatuximab), the carfilzomib, and the dexamethasone is provided as a separate pharmaceutical composition. In some embodiments, the pharmaceutical compositions and formulations further comprise a pharmaceutically acceptable carrier.

In some embodiments, an anti-CD38 antibody described herein (such as isatuximab) is in a formulation comprising about 20 mg/mL (500 mg/25 mL) antibody, about 20 mM histidine, about 10% (w/v) sucrose, about 0.02% (w/v) polysorbate 80 at pH 6.0. In some embodiments, an anti-CD38 antibody described herein (such as isatuximab) is in a formulation comprising about 20 mg/mL antibody, about 100 mg/mL sucrose, 2.22 mg/mL histidine hydrochloride monohydrate, about 1.46 mg/ml histidine, and about 0.2 mg/ml polysorbate 80. In some embodiments, the formulation comprises water for injection (WFI), such as sterile water for injection (SWFI). In some embodiments, the formulation is sterile. In some embodiments, a single use of the formulation comprises 5 ml of the formulation (i.e., 100 mg anti-CD38 antibody). In some embodiments, the single use 5 ml formulation is provided in, e.g., a type 16 mL colorless clear glass vial fitted with elastomeric closure. In some embodiments, the fill volume of the vial has been established to ensure removal of 5 mL. In some embodiments, the fill volume is 5.4 mL. In some embodiments, a single use of the formulation comprises 25 ml of the formulation (i.e., 500 mg anti-CD38 antibody). In some embodiments, the single use 25 ml formulation is provided in, e.g., a 30 mL colorless clear glass vial fitted with elastomeric closure. In some embodiments, the fill volume of the vial has been established to ensure removal of 25 mL. In some embodiments, the formulation is stable for at least about 6, 12, 18, 24, 30, or 36 months, including any range in between these values, at a temperature between about 2° C. and about 8° C. and protected from light. In some embodiments, the formulation is diluted for infusion in 0.9% sodium chloride or 5% dextrose. In some embodiments, the diluted infusion solution is stable for up to about 6, 12, 18, 24, 30, 36, 42, or 48 hours, including any range in between these values, between about 2° C. and about 8° C. In some embodiments, the diluted solution for infusion is stable following storage between about 2° C. and about 8° C. for a further 8 hours (including the infusion time) at room temperature. In some embodiments, the diluted solution for infusion is stable in the presence of light. In some embodiments the bag in which the diluted solution for infusion is stored is fabricated from polyolefins (PO), polyethylene (PE), polypropylene (PP), polyvinyl chloride (PVC) with di(ethylhexyl)phthalate (DEHP) or ethyl vinyl acetate (EVA). In some embodiments, the tubing used for infusion is fabricated from PE, PVC (with or without DEHP), polybutyldiene (PBD), or polyurethane (PU) with an in-line filter (polyethersulfone (PES), polysulfone or nylon).

### Methods of Treatment

Provided herein are methods for treating or delaying progression of multiple myeloma in an individual (e.g., a human individual) comprising administering to the individual an effective amount of an anti-CD38 antibody (e.g., an anti-CD38 antibody comprising (a) a heavy chain variable domain (V_{H}) that comprises: a CDR-H1 comprising the amino acid sequence DYWIVIQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody is isatuximab.

In some embodiments, the method comprises administering isatuximab to the individual at a dose of 10 mg/kg on Days 1, 8, 15, and 22 of a first 28-day cycle; administering the isatuximab at a dose of 10 mg/kg on Days 1 and 15 of a 28-day cycle for at least 11 cycles; and administering the isatuximab at a dose of 10 mg/kg once every 28 days of one or more additional 28-day cycles following the at least 11 cycles. In some embodiments, the method comprises administering the isatuximab at a dose of 10 mg/kg on Days 1 and 15 of a 28-day cycle for at least 23 cycles, and administering the isatuximab at a dose of 10 mg/kg once every 28 days of one or more additional 28-day cycles following the at least 23 cycles. In some embodiments, the treatment extends the progression-free survival (PFS) of the individual.

In some embodiments, the method comprises administering the anti-CD38 antibody (e.g., isatuximab) to the individual at a dose of 10 mg/kg on Days 1, 8, 15, and 22 (e.g., qw) of a first 28-day cycle; administering the isatuximab at a dose of 10 mg/kg on Days 1 and 15 (e.g., q2w) of one or more 28-day cycles following the first 28-day cycle; measuring the individual's serum and urine M-protein levels at one or more time point during the one or more 28-day cycles following the first 28-day cycle; and administering the isatuximab at a dose of 10 mg/kg on Day 1 (e.g., q4w) of one or more additional 28-day cycles when or after the individual's serum and urine M-protein levels are detectable by immunofixation but not on electrophoresis at the one or more time points. Method of measuring serum and urine M-protein levels are well known in the art and described in, e.g., Jenkins (2009) Clin Biochem Rev. 30(3): 119-122; Leung, Nelson “Chapter 8: Clinical Tests for Monoclonal Proteins.” Onco-Nephrology Curriculum, American Society of Nephrology 2016, pages 1-5). In some embodiments, the individual's serum and urine M-protein levels are detectable by immunofixation but not on electrophoresis for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months after the one or more time points. In some embodiments the isatuximab is administered once every 28 days of one or more 28-day cycles when or after the individual's serum and urine M-protein levels are detectable by immunofixation but not on electrophoresis for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months after the one or more time points. In some embodiments, the method comprises administering the anti-CD38 antibody (e.g., isatuximab) to the individual at a dose of 10 mg/kg on Days 1, 8, 15, and 22 (e.g., qw) of a first 28-day cycle; administering the anti-CD38 antibody (e.g., isatuximab) at a dose of 10 mg/kg on Days 1 and 15 (e.g., q2w) of one or more 28-day cycles after the first 28-day cycle until the individual's serum and urine M-protein levels are detectable by immunofixation but not on electrophoresis, and administering the anti-CD38 antibody (isatuximab) on Day 1 (e.g., q4w) of every 28-day cycle when or after the individual's serum and urine M-protein levels are determined to be detectable by immunofixation but not on electrophoresis. In some embodiments, the individual's serum and urine M-protein levels are detectable by immunofixation but not on electrophoresis for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles when or after the individual's serum and urine M-protein levels are detectable by immunofixation but not on electrophoresis for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some isatuximab is administered at a dose of 10 mg/kg on Days 1 and 15 of one or more 28 day cycles for at least 11 cycles prior to administering the isatuximab once every 28 days of one or more 28-day cycles. In some isatuximab is administered at a dose of 10 mg/kg on Days 1 and 15 of one or more 28 day cycles for at least 23 cycles prior to administering the isatuximab once every 28 days of one or more 28-day cycles. In some embodiments, the treatment extends the progression free survival (PFS) of the individual.

In some embodiments, the method comprises measuring the individual's serum M-protein at a first time point prior to administration of the anti-CD38 antibody (e.g., isatuximab); administering isatuximab to the individual at a dose of 10 mg/kg on Days 1, 8, 15, and 22 (e.g., qw) of a first 28-day cycle; administering the anti-CD38 antibody (e.g., isatuximab) at a dose of 10 mg/kg on Days 1 and 15 (e.g., q2w) of one or more 28-day cycles after the first 28-day cycle; measuring the individual's serum M-protein at a second time point during at least one or more 28-day cycles after the first 28-day cycle; and administering the anti-CD38 antibody (e.g., isatuximab) at a dose of 10 mg/kg on Day 1 (e.g., q4w) of the one or more additional 28-day cycles if (a) the individual's serum M-protein level at the second time point is reduced by at least 90% as compared to the individual's serum M-protein level at the first time point and (b) the individual's urine M-protein level at the second time point is less than 100 mg/24 hours. In some embodiments, the reduction in the individual's serum M-protein level and the individual's urine M-protein level of less than 100 mg/24 hours are maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles after the reduction in the individual's serum M-protein level and the individual's urine M-protein level of less than 100 mg/24 hours is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments, the method comprises measuring the individual's serum M-protein level prior to the administration of the anti-CD38 antibody (e.g., isatuximab); administering the anti-CD38 antibody (e.g., isatuximab) to the individual at a dose of 10 mg/kg on Days 1, 8, 15, and 22 (e.g., qw) of a first 28-day cycle; administering the anti-CD38 antibody (e.g., isatuximab) at a dose of 10 mg/kg on Days 1 and 15 (e.g., q2w) of one or more 28-day cycles after the first 28-day cycle until (a) the individual's serum M-protein level at reduced by at least 90% as compared to the serum M-protein level prior to the administration of isatuximab and (b) the individual's urine M-protein level at the second time point is less than 100 mg/24 hours; and administering the anti-CD38 antibody (e.g., isatuximab) at a dose of 10 mg/kg on Day 1 (e.g., q4w) of every 28-day cycle after (a) the individual's serum M-protein level is determined to be reduced by at least 90% as compared to the individual's serum M-protein level at the first time point and (b) the individual's urine M-protein level is determined to be less than 100 mg/24 hours. In some embodiments, the reduction in the individual's serum M-protein level and the individual's urine M-protein level of less than 100 mg/24 hours are maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles when or after the reduction in the individual's serum M-protein level and the individual's urine M-protein level of less than 100 mg/24 hours is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some isatuximab is administered at a dose of 10 mg/kg on Days 1 and 15 of one or more 28 day cycles for at least 11 cycles prior to administering the isatuximab once every 28 days of one or more 28-day cycles. In some isatuximab is administered at a dose of 10 mg/kg on Days 1 and 15 of one or more 28 day cycles for at least 23 cycles prior to administering the isatuximab once every 28 days of one or more 28-day cycles. In some embodiments, the treatment extends the progression free survival (PFS) of the individual.

In some embodiments, the method comprises administering the anti-CD38 antibody (e.g., isatuximab) to the individual at a dose of 10 mg/kg on Days 1, 8, 15, and 22 (e.g., qw) of a first 28-day cycle; administering the anti-CD38 antibody (e.g., isatuximab) at a dose of 10 mg/kg on Days 1 and 15 (e.g., q2w) of one or more 28-day cycles after the first 28-day cycle until the individual achieves a response of at least VGPR (“very good partial response”) and administering the anti-CD38 antibody (e.g., isatuximab) at a dose of 10 mg/kg on Day 1 (e.g., q4w) of every 28-day cycle when or after the individual achieves at least VGPR. In some embodiments, the individual achieves at least stable VGPR. In some embodiments, stable VGPR refers to VGPR that is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles when or after the response of at least VGPR is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments, VGPR is assessed according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473, the contents of which are incorporated herein by reference in their entireties. (See also Table A.) In some embodiments, the method comprises administering isatuximab to the individual at a dose of 10 mg/kg on Days 1, 8, 15, and 22 of a first 28-day cycle; administering the isatuximab at a dose of 10 mg/kg on Days 1 and 15 of one or more 28-day cycles after the first 28-day cycle until the individual achieves a response of at least very good partial response (VGPR); and administering the isatuximab at a dose of 10 mg/kg once every 28 days of one or more additional 28-day cycles when or after the individual achieves the response of at least VGPR. In some embodiments, the individual achieves at least stable VGPR. In some embodiments, stable VGPR refers to VGPR that is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles when or after the response of at least VGPR is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments, the method comprises administering the isatuximab to the individual at a dose of 10 mg/kg on Days 1, 8, 15, and 22 of first 28-day cycle; administering the isatuximab at a dose of 10 mg/kg on Days 1 and 15 of one or more 28-day cycles after the first 28-day cycle; measuring the individual's response to the treatment at one or more time points during the one or more 28-day cycles after the first 28-day cycle and selecting individuals who have at least a Very Good Partial Response (VGPR); and administering the isatuximab at a dose of 10 mg/kg once every 28 days of one or more additional 28-day cycles to the selected individuals. In some embodiments, the individual achieves at least stable VGPR. In some embodiments, stable VGPR refers to VGPR that is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some embodiments the isatuximab is administered once every 28 days of one or more 28-day cycles when or after the response of at least VGPR is maintained for at least about any one of 1, 2, 3, or 4 weeks, or at least about any one of 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 months. In some isatuximab is administered at a dose of 10 mg/kg on Days 1 and 15 of one or more 28 day cycles for at least 11 cycles prior to administering the isatuximab once every 28 days of one or more 28-day cycles. In some isatuximab is administered at a dose of 10 mg/kg on Days 1 and 15 of one or more 28 day cycles for at least 23 cycles prior to administering the isatuximab once every 28 days of one or more 28-day cycles. In some embodiments, the treatment extends the progression free survival (PFS) of the individual.

In some embodiments, the multiple myeloma is smoldering multiple myeloma (SMM). In some embodiments, the multiple myeloma is newly diagnosed multiple myeloma. In some embodiments, the multiple myeloma is relapsed and/or refractory multiple myeloma (RRMM). In some embodiments, the individual received 1, 2, or 3 prior therapies for multiple myeloma. In some embodiments, the individual received more than three prior therapies with multiple myeloma. In some embodiments, the individual received prior therapy with a proteasome inhibitor. In some embodiments, the individual received prior therapy with an immunomodulatory agent.

In some embodiments, the anti-CD38 antibody (e.g., isatuximab) is administered in conjunction with at least one additional agent. In some embodiments, the least one additional agent comprises an immunomodulatory drug. In some embodiments, the immunomodulatory drug is thalidomide, lenalidomide or pomalidomide. In some embodiments, the at least one additional agent comprises a proteasome inhibitor. In some embodiments, the proteasome inhibitor is bortezomib, carfilzomib, marizomib, oprozomib, and ixazomib. In some embodiments, the at least one additional agent comprises a corticosteroid. In some embodiments, the corticosteroid is dexamethasone.

### Articles of Manufacture or Kits

In another embodiment of the invention, an article of manufacture or a kit is provided comprising an anti-CD38 antibody (such as isatuximab). In some embodiments, the article of manufacture or kit further comprises at least one additional agent (e.g., one or more additional agents described herein). In some embodiments, the article of manufacture or kit further comprises package insert comprising instructions for using the anti-CD38 antibody (e.g., isatuximab) according to a method described herein to treat or delay progression of multiple myeloma (e.g., smoldering multiple myeloma, newly-diagnosed multiple myeloma, refractory multiple myeloma, or relapsed and refractory multiple myeloma).

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Model Based Approach to Evaluate Isatuximab Monthly Dosing Regimen in Relapsed/Refractory Multiple Myeloma Patients

### Background

Isatuximab (Isa) is a CD38 monoclonal antibody with multiple modes of action for killing tumor cells through direct tumor targeting and immune cell engagement (Moreno et al. (2019) Clin Cancer Res. 25(10): 3176-3187). The addition of Isa to pomalidomide (P) and dexamethasone (d) was associated with a significant and clinically meaningful benefit in progression-free survival (PFS) in heavily pre-treated patients with relapsed/refractory multiple myeloma (RRMM) (Attal et al. (2019) Lancet, 394(10214): 2096-2107). Isa, in combination with Pd, is approved in the United States, the European Union, Canada, Australia, Switzerland, and Japan for the treatment of adult patients with RRMM who have received at least two prior therapies including lenalidomide and a proteasome inhibitor.

### Aim

The objectives of this Example were to characterize the relationship between serum M-protein kinetics and PFS in RRMM patients using data from the Phase 3 clinical trial of isatuximab in combination with pomalidomide and dexamethasone discussed above (“Isa-Pd trial”) and to simulate longitudinal serum M-protein and PFS assess when to switch isatuximab treatment from Q2W to monthly dosing in a manner that preserves clinical benefit, for example as measured by length of Progression Free Survival.

### Methods

A joint model of serum M-protein dynamics and PFS was developed using data from 256 evaluable patients from the Isa-Pd trial. Patients received Isa intravenously at 10 mg/kg once weekly (QW) for 4 weeks, then every other week (Q2W) for 28-day cycles in combination with standard Pd (Isa-Pd) or Pd alone in the control arm. A tumor growth inhibition model was used to describe the serum M-protein kinetics under treatment effects of Isa-Pd or Pd alone, in which Isa exposure was predicted using individual PK parameters obtained from the population PK analysis (Fau et al. “Pharmacokinetic time-dependency and covariates modelling of Isatuximab monoclonal antibody in multiple myeloma patients: analysis from pooled phase I/II & phase III studies, Population Approach Group in Europe 2019 Meeting, Stockholm, Sweden, Jun. 11-14, 2019, Abstract 8956) and Pd exposure was predicted from K-PD model using dosing history. Trial simulations were then performed using individual PK/PD parameters of patients from the Isa-Pd trial to evaluate whether efficacy is maintained after switching to a monthly dosing regimen.

### Results

The joint model identified the instantaneous changes (slope) in serum M-protein as the best on-treatment predictor for PFS and also identified baseline patient characteristics impacting serum M-protein kinetics (serum albumin and serum β2 microglobulin on the baseline serum M-protein levels and the non-IgG type on the serum M-protein growth rate, the serum M-protein slope), and PFS (presence of plasmacytomas). Non-IgG MM patients have similar behavior on serum M-protein kinetics for the first 60 weeks even with higher exposure and similar progression free survival compared to IgG MM patients supporting the non-dose adjustment based on IgG status. Clinical trial simulation of the regimen used in the Phase 3 Isa-Pd trail demonstrated that switching all patients on treatment at 6 months to a monthly Isa regimen would shorten the median time to progression (TTP) (i.e. increase in serum M-protein greater than 25% and an absolute increase greater than 5 g/L compared to nadir) by 4.1 weeks and would shorten median PFS by 2.3 weeks (from 14.03 to 13.45 months). Based on TTP criteria, patients with no risk of earlier progression (57.7%) due to 6 months switch tend to have lower baseline tumor burden (lower serum M-protein and lower percent of bone marrow plasma cell) and better prognostic factors (higher glomerular filtration rate, higher albumin, lower β2 microglobulin). At 6 months, 85% of these patients had predicted stable “at least” VGPR status.

conclusions

Trial simulations supported the choice of the approved isatuximab 10 mg/kg QW/Q2W regimen and showed that switching to a monthly Isa regimen after 6 months may reduce clinical benefit in overall population. However, a subpopulation of patients with good prognosis and obtaining stable at least VGPR status by 6 months may be able to switch to a monthly regimen after 6 months without compromising disease progression risk. Model-based drug development has been successfully applied to support treatment decisions in RRMM patients.

### Example 2: Joint Modeling and Simulation of M-Protein Dynamics and Progression-Free Survival for Alternative Isatuximab Dosing with Pomalidomide/Dexamethasone

### a) Introduction

Despite significant advances and prolongation in overall survival (OS), multiple myeloma (MM) remains incurable, with the majority of patients relapsing and requiring additional treatment [1]. Isatuximab is an immunoglobin G1 (IgG1) monoclonal antibody that targets the CD38 transmembrane glycoprotein in MM. Isatuximab kills tumor cells via multiple biological mechanisms, including antibody-dependent cell-mediated cytotoxicity, complement-dependent cytotoxicity, direct induction of apoptosis without cross-linking, and inhibition of CD38 enzymatic activity. In a Phase 1b study of patients with relapsed/refractory MM (RRMM), a 10 mg/kg once/twice weekly (QW-Q2W) dose of isatuximab (Isa) in combination with pomalidomide (P) and low-dose dexamethasone (d, Isa-Pd) achieved an overall response rate (ORR) of 64.5% and median progression-free survival (PFS) of 17.6 months. These results, combined with exposure-response and disease modeling of tumor burden (serum M-protein), provided the justification for the 10 mg/kg QW-Q2W of Isa-Pd [2,3]. This combination was then assessed in the Phase 3 ICARIA-MM study, which showed that addition of isatuximab to Pd significantly improved PFS in RRMM patients [4]. Based on this pivotal study, isatuximab in combination with Pd is approved in multiple countries for RRMM patients with ≥2 prior treatment lines, including lenalidomide and a proteasome inhibitor. Furthermore, to date, isatuximab in combination with carfilzomib/dexamethasone is approved in the United States for relapsed MM patients with 1-3 prior treatment lines, and in the European Union for MM patients with ≥1 prior therapy, based on the Phase 3 IKEMA study [5-7].

Efforts have been made to develop tumor growth inhibition (TGI) models and predict clinical responses, overall survival (OS), or PFS rates in cancer patients from various clinical settings [8,9]. TGI models are used to find early changes in tumor size that would predict OS or PFS. Joint models have emerged as a promising framework for concurrently investigating the relationship between continuous disease progression through longitudinal outcomes such as biomarkers, tumor size, and the incidence of clinical events such as progression and death. These models provide precise and unbiased estimation of the parameters in an informative censoring context [10]. Mechanistic joint models predicted OS in clinical trials for atezolizumab in urothelial carcinoma, cabazitaxel in metastatic prostate cancer, and aflibercept in metastatic colorectal cancer [11-13].

In most patients, MM is characterized by the secretion of a monoclonal Ig protein (M-protein) called paraprotein, which is produced by the abnormal plasma cells. Similar to tumor burden for solid tumors, serum M-protein levels are part of the response criteria for MM patients [14] and thus their dynamic change can predict long-term clinical benefit (PFS, OS). Several examples in MM showed that TGI modeling based on longitudinal M-protein can be used to predict OS or PFS [15-18].

For isatuximab, the joint modeling framework was used to integrate early drug development results with later-stage clinical data from Phase ½ monotherapy and Phase 1 combination studies [2,3,19]. Disease progression was initially captured together with serum M-protein dynamics using a joint model and accounting for dropout. Longitudinal serum M-protein modeling provided more insights in patient response over time and supported Phase 2 and Phase 3 dosing-regimen selection in MM patients. This framework and modelling approach can be extended to account for PFS, and therefore improves the predictive and simulation value of the models in exploring the benefits of a different dosing strategy.

Objectives of this work were therefore to (i) quantitatively evaluate the association between serum M-protein kinetics, baseline covariates, and PFS in RRMM patients in both Isa-Pd and Pd arms of the ICARIA-MM study, and (ii) simulate longitudinal serum M-protein and PFS when switching to a hypothetical monthly isatuximab dosing regimen after 6 months.

### b) Materials and Methods

Study Design and Data

Data were obtained from the Phase 3 ICARIA-MM study. Isatuximab was administered intravenously at 10 mg/kg QW for 4 weeks followed by bi-weekly for 28-day cycles in combination with standard pomalidomide (4 mg orally on days 1-21 in each cycle) and dexamethasone (40 or 20 mg for patients aged ≥75 years orally or intravenously on days 1, 8, 15, 22 in each cycle). The study was conducted following the principles of the Declaration of Helsinki and ICH GCP Guidelines. The protocol was approved by institutional review boards and independent ethics committees at the participating institutions. All patients provided written informed consent. Primary study endpoint was PFS. Response and disease progression were determined by an independent response committee using the International Myeloma Working Group (IMWG) criteria, based on central, M-protein laboratory assessments and radiology review [14]. Patients with serum M-protein values, including one baseline value and for whom response could be evaluated by serum M-protein were included in the analysis. Serum M-protein was assessed by a hybrid assay using immunocapture and liquid chromatography coupled with high-resolution mass spectrometry. Per protocol, serum M-protein was measured at baseline, end of each cycle, and study end.

Model Development

Serum M-protein longitudinal data from both study arms and PFS data were first modeled separately. Treatment exposure over time was introduced in the longitudinal model using the concentrations predicted by the individual PK parameters for isatuximab and a kinetic-pharmacodynamic (K-PD) model for pomalidomide and dexamethasone. Several joint models were then used to find the best link between serum M-protein kinetics and PFS.

Population PK Model for Isatuximab

A two-compartment PK model with parallel linear and nonlinear (Michaelis-Menten) elimination from the central compartment and time-varying linear clearance function was used to describe the plasma concentrations of isatuximab versus time data collected from four Phase 1-3 clinical trials including ICARIA-MM [20]. The equations of this structural PK model are presented in Example 2A. Individual PK parameters for ICARIA-MM patients were obtained as post-hoc estimates and typical PK parameters were attributed for patients without PK data.

K-PD Model for Pomalidomide and Dexamethasone

Since concentrations of combined Pd were not measured in this study, the kinetics of these drugs were simplified using a K-PD modeling approach [21]. Their PK was therefore described by a simple, virtual one compartment with bolus input and fixed elimination-rate constant derived from their central distribution volume and clearance value estimates in the literature [22,23].

TGI Model for M-Protein Data and Covariate Selection

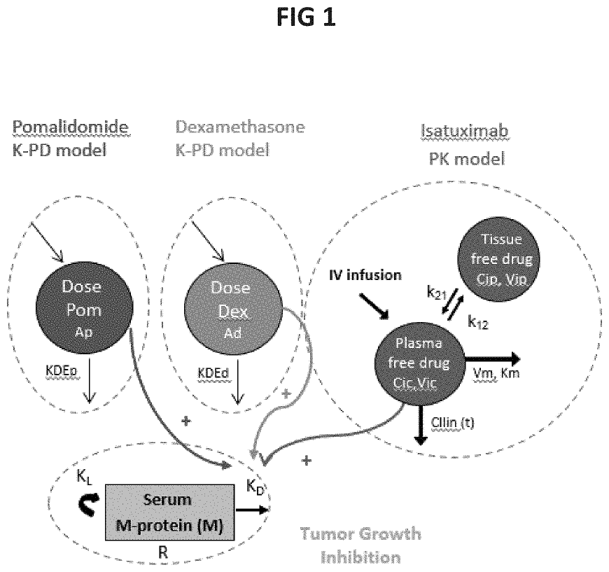

A TGI model accounting for the dynamics of tumor growth, antitumor drug effect, and resistance to drug effect was developed by Claret et al. [24]. This model was also successfully applied in the literature to describe serum M-protein data as a surrogate of tumor growth in MM patients [15,16,18,25,26]. In this analysis, a mechanism-based model drawn from the Claret's TGI model was proposed to describe the underlying disease progression and exposure-driven drug effect of isatuximab and Pd on the serum M-protein time-course. The structural model for this TGI model, shown in FIG. 1, is described by the following differential equation:

$$\frac{d M}{d t} = K L * M - K {D}_{i} * {e}^{- {R}_{i} * t} * {C}_{i M} * M - K {D}_{pd} * {e}^{- {R}_{pd} * t} * ( {C}_{p M} + {C}_{dM} ) * M ; M ( t = 0 ) = M 0$$

where M is serum M-protein at time t, M₀ the baseline serum M-protein, K_{L} the tumor growth rate, KDᵢ and KD_{pd} the shrinkage rate due to isatuximab and combined Pd exposure respectively, Rᵢ and R_{pd} the rate constant of resistance appearance to isatuximab and combined Pd, respectively, and C_{iM}, C_{pM} and C_{dM} are the molar concentration of isatuximab, pomalidomide, and dexamethasone at time t, respectively. The contribution of C_{pM} and C_{dM} in increasing M-protein shrinkage rate KD_{pd} was assumed to be equal, based on the response rates of a randomized Phase 2 study comparing pomalidomide alone or combined with dexamethasone [27].

An exponential interindividual model implying a log-normal distribution was included on all parameters. The variance-covariance matrix was modeled using a diagonal matrix. The residual variability was modeled using a combined additive and proportional model.

The covariate analysis was performed after obtaining the base model. Twenty-six baseline covariates were tested: demographics, baseline laboratory measurements, and disease-related patient characteristics. See Table B below. In case of missing data, the median value was input for continuous covariates; missing was considered as an additional category for categorical covariates. The parameter-covariate relationship was first explored graphically using individual parameter estimates. The Conditional Sampling for Stepwise Approach based on Correlation tests (COSSAC) covariate selection algorithm was then used for automatic building of the covariate model [28,29]. The best covariate model was selected using the corrected version of Bayesian Information Criteria (BICc) [30]. In addition, only significant covariates with Wald-test p-value <0.05 were kept in the final model.

TABLE B
List of baseline patient characteristics tested as covariates

| Baseline covariate | Label |
|---|---|
| AGE | Age |
| HT | Height |
| WT | Weight at baseline |
| SEX | Gender |
| RACE | Race |
| GFR | Creatinine clearance by MDRD |
| | equation at baseline |
| ALB | Albumin |
| ALK | Serum alkaline phosphatase |
| AST | Aspartate aminotransferase |
| ALT | Alanine aminotransferase |
| BIL | Total bilirubin |
| LDH | Lactate dehydrogenase |
| B2MG | Beta-2 microglobulin |
| BMPC | Bone marrow plasma cells at baseline |
| MPROT | Serum M-protein at baseline |
| CCAL | Corrected serum calcium |
| CYTO | High-risk cytogenetics |
| ECOG | Performance status |
| ISS | International staging system at study entry |
| R-ISS | Revised-ISS at study entry |
| PCYTOMA | Plasmacytoma |
| IGTYPE | Main type of immunoglobulin |
| DURATION | Time from diagnosis to first dose |
| LINE | Number of treatment lines |
| REFA_LEN | Refractory to lenalidomide |
| REFA_PI | Refractory to proteasome inhibitor |

PFS Model and Covariate Selection

PFS was modeled using a parametric proportional-hazard model with log-logistic distribution for baseline hazard:

$${h}_{0} ( t ) = \frac{\frac{s}{T e} {( \frac{t}{T e} )}^{s - 1}}{1 + {( \frac{t}{T e} )}^{s}}$$

where Te is the scale parameter (characteristic time) and s the shape parameter. Exponential and Weibull distribution were also tested. The baseline covariates were tested as potential prognostic factors using the classical stepwise covariate modeling method. The same criteria for covariate selection in the longitudinal M-protein model development were used.

Joint Modeling of Serum M-Protein and PFS

Longitudinal and PFS models were built separately; thereafter several joint models were used to find the best link between serum M-protein kinetics and PFS (including no link, current serum M-protein, and current M-protein slope and AUC). Significant covariates found in the longitudinal and PFS submodels were evaluated, and only significant covariates with the Wald-test were kept in the joint model.

Parameter Estimation

Parameter estimation of all models was performed using the Stochastic Approximation Expectation Maximization (SAEM) algorithm implemented in the software Monolix v.2019R1. Data below limit of quantification (LOQ) for serum M-protein were taken into account using the extended SAEM algorithm implemented in Monolix.

Model Selection and Evaluation

Model selection was based on BIC and the model giving the lowest BIC was retained. Model evaluation was performed by investigating both residual- and simulation-based diagnostics, including the Individual Weighted Residuals (IWRES), visual predictive checks (VPC) for the longitudinal part, Cox-Snell and deviance residuals [31], de-trended prediction discrepancies [32], and Kaplan Meier VPC for PFS, respectively. Additional goodness-of-fit plots were assessed by visual inspection of individual fits or by comparing observations versus individual predictions. Longitudinal VPC accounted for risk of progression using the methods described by Friberg et al. [33]. Briefly, it involved reproducing the event mechanisms in simulation and omitting simulations occurring after a simulated progression time. PFS VPC considered the design of each patient, i.e. dose regimens and follow-up duration. Indeed, the simulated time to progression (TTP) was censored by the maximum time between duration of follow-up, end of treatment, and observed TTP.

Simulation of Monthly Dosing Regimen

To evaluate longitudinal serum M-protein and PFS after switching to a hypothetical monthly isatuximab dosing regimen after 6 months, 1000 trials were simulated with both the Isa-Pd and Pd arms for 80 weeks. Patients received isatuximab 10 mg/kg QW for 4 weeks then Q2W for 20 weeks, then monthly in the Isa-Pd arm. The combination dosing regimen with standard Pd was the same as in ICARIA-MM. Patients at risk at 6 months were evaluated for the impact on TTP (increase >25% with absolute change of ≥5 g/L for serum M-protein compared to nadir) and PFS. The original ICARIA-MM Isa-Pd arm was also simulated with patients receiving isatuximab 10 mg/kg QW-Q2W, to present results as median (5^{th}-95^{th} VD percentiles) difference from the original arm. Hazard ratios (HR) for two regimens vs control arm were also compared.

### c) Results

Data Used for Model Building

Among the 307 randomized patients in the ICARIA-MM trial, 256 serum M-protein evaluable patients (128 patients/arm) were considered in this analysis. In this serum M-protein population (N=256), median PFS was significantly longer with Isa-Pd vs Pd (11.4 months [95% CI 8.5-13.8] vs 6.96 months [95% CI 4.4-8.5]; HR 0.618, 95% CI 0.44-0.87; p=0.0048). Similar observations on PFS and HR were obtained for the overall population (N=307), although 16.6% of ICARIA-MM patients could not be included in this analysis.

Baseline patient characteristics were balanced across arms. See Table C. Median age was 67 years (50% female). Baseline, median serum β2-microglobulin and serum albumin were 3.5 mg/L and 0.67 g/L, respectively. High-risk cytogenetics were present in 53 (21%) patients and median estimated glomerular filtration rate (e-GFR) was 70 mL/min. Most patients were IgG MM type (190 [74%]), had no plasmacytomas (232 [91%]), and 64 (25%) and 164 (64%) patients had Revised International Staging System (R-ISS) I or II stage at diagnosis, respectively. Baseline median serum M-protein was 23 g/L with a wide range of values (5-95 g/L), together with various profiles during treatment. A total of 2637 serum M-protein measurements in the 256 evaluable patients were considered, with a median of 14 (range 2-22) assessments/patient. The data below LOQ accounted for 14% (22% in the Isa-Pd, 6% in the Pd arm).

TABLE C
Baseline demographics and patient
characteristics in serum M-protein population

| | Isatuximab plus | | | |
|---|---|---|---|---|
| | pomalidomide and | | Pomalidomide and | |
| | dexamethasone | | dexamethasone | |
| | (Isa-Pd) | | (Pd) | |
| | (n = 128) | | (n = 128) | |
| Age, years (range) | 68 | (36-83) | 66 | (41-86) |
| Sex, n (%) | | | | |
| Female | 56 | (44) | 72 | (56) |
| Male | 72 | (56) | 56 | (44) |
| Weight (kg), median (range) | 74 | (34-110) | 73 | (39-140) |
| eGFR (mL/min), median (range) | 69 | (30-177) | 71 | (31-135) |
| R-ISS, n (%) | | | | |
| I | 36 | (28) | 28 | (22) |
| II^{a} | 81 | (63) | 83 | (65) |
| III | 11 | (9) | 17 | (13) |
| Serum β2-microglobulin | 3.5 | (1.1-27) | 3.5 | (0.7-55) |
| (mg/L), median (range) | | | | |
| Serum albumin (g/L), | 0.67 | (0.29-0.89) | 0.67 | (0.30-0.84) |
| median (range) | | | | |
| Serum M-protein at | 22 | (5-95) | 23 | (5-83) |
| baseline (g/L), median (range) | | | | |
| Type of myeloma, n (%) | | | | |
| IgG | 97 | (76) | 93 | (73) |
| Non-IgG | 31 | (24) | 35 | (27) |
| Plasmacytomas, n (%) | | | | |
| Yes | 13 | (10) | 11 | (9) |
| No | 115 | (90) | 117 | (91) |
| Cytogenetic risk at | | | | |
| study entry, n (%) | | | | |
| Standard | 86 | (67) | 63 | (49) |
| High | 20 | (16) | 33 | (26) |
| Missing | 22 | (17) | 32 | (25) |

e-GFR, estimated glomerular filtration rate; R-ISS, Revised Multiple Myeloma International Staging System (derived based on the combination of serum β2-microglobulin, albumin, cytogenetic risk, and lactate dehydrogenase).
^{a}As pre-specified in the study statistical analysis plan, patients with unknown cytogenetics at baseline were classified as R-ISS stage II.

Modeling Serum M-Protein Kinetics and PFS

The proposed TGI model provided an adequate fit for the longitudinal serum M-protein data of both study arms. It performed better than the Wang model [34]. In addition, the fit was improved when adding the PK of isatuximab compared to a K-PD model only. Twenty-six potential covariates were evaluated by testing their relationship with all the longitudinal model parameters. The final longitudinal model includes three covariates: the effect of baseline serum albumin and β2-microglobulin on baseline serum M-protein levels, and the non-IgG type on KL, the serum M-protein growth rate. Patients with low baseline albumin and high β2-microglobulin levels were more likely to have higher serum M-protein at baseline. Of note, these laboratory tests are part of the ISS and R-ISS and are relevant for prognosis assessment. Non-IgG MM patients tend to have a more rapid tumor regrowth (i.e. faster re-increase in serum protein levels) compared with IgG MM patients.

Regarding PFS, a log-logistic model best characterized the underlying baseline hazard distribution. Baseline covariates such as presence of plasmacytoma, serum albumin, and serum M-protein were significant (p<0.005). Patients with high baseline serum M-protein, low baseline albumin and presence of plasmacytomas had lower median PFS. Further information about the modeling results of longitudinal data and PFS is included in Tables B and D.

TABLE D
Parameter estimates of the longitudinal model without covariates and with significant
covariates. P-values of covariate effects were computed by Wald test.

| | | | Covariate model | | |
|---|---|---|---|---|---|
| | Basic model | | | RSE | P-value |
| Fixed parameters | Estimate | RSE (%) | Estimate | (%) | (Wald-test) |
| M0 (g/L) | 18.7 | 3.91 | 17.4 | 3.33 | |
| β1~ALBN | | | −1.39 | 16.1 | 4.81E−10 |
| β2~B2MG | | | 0.333 | 16.9 | 3.60E−09 |
| KL (day⁻¹) | 0.00562 | 8.17 | 0.00453 | 9.31 | |
| β3~Non_IgG | | | 0.672 | 26.1 | 0.000128 |
| KDi (L · mol⁻¹ · day⁻¹) | 0.0139 | 12.8 | 0.015 | 15.2 | |
| Ri (day⁻¹) | 0.00936 | 22.2 | 0.0148 | 27.4 | |
| KDpd (L · mol⁻¹ · | 0.176 | 7.52 | 0.178 | 7.49 | |
| day⁻¹) | | | | | |
| Rpd (day⁻¹) | 0.0104 | 8.21 | 0.011 | 7.89 | |
| Interindividual | | | | | |
| variability | | | | | |
| ω_M0 (%) | 61.3 | 4.59 | 50.4 | 4.66 | |
| ω_KL (%) | 102 | 5.93 | 102 | 6.31 | |
| ω_Kdi (%) | 100 | 11.3 | 97.3 | 11.8 | |
| ω_Ri (%) | 174 | 12.3 | 202 | 13.3 | |
| ω_KDpd (%) | 89.8 | 6.52 | 92.4 | 6.56 | |
| ω_Rpd (%) | 92.9 | 7.75 | 84.3 | 7.35 | |
| Residual variability | | | | | |
| σ additive (g/L) | 0.394 | 7.82 | 0.367 | 7.24 | |
| σ proportional (%) | 15.2 | 3.35 | 15.7 | 3.17 | |

ALBN, baseline serum albumin normalized to the upper limit value;
B2MG, baseline β2-microglobulin;
M0, serum M-protein at baseline;
RSE, relative standard error.

TABLE E
Parameter estimates of joint model with different links.

| Model | No link | | Current M | | Slope M | |
|---|---|---|---|---|---|---|
| Statistical criteria | | | | | | |
| −2LL | | 14460.82 | | 14349.28 | | 14256.76 |
| BIC | | 14591.25 | | 14487.77 | | 14395.25 |
| Fixed parameters | Estimate | RSE (%) | Estimate | RSE (%) | Estimate | RSE (%) |
| M0 (g/L) | 17.3 | 3.31 | 17.3 | 3.3 | 17 | 3.33 |
| β~ALBN | −1.38 | 16 | −1.38 | 16 | −1.41 | 15.7 |
| β2~B2MG | 0.338 | 16.5 | 0.331 | 16.8 | 0.329 | 17 |
| KL (day⁻¹) | 0.005 | 9.86 | 0.00453 | 9.79 | 0.00644 | 8.4 |
| β3~Non_IgG | 0.553 | 32 | 0.608 | 29.8 | 0.608 | 24.4 |
| KDi (L · mol⁻¹ · day⁻¹) | 0.0201 | 15.3 | 0.0128 | 11 | 0.018 | 12.8 |
| Ri (day⁻¹) | 0.0136 | 24.4 | 0.013 | 22.2 | 0.00891 | 23.7 |
| KDpd (L ·mol^{-‘}·day⁻¹) | 0.157 | 8.21 | 0.179 | 7.68 | 0.175 | 7.16 |
| Rpd (day⁻¹) | 0.0096 | 8.83 | 0.0104 | 8.16 | 0.00799 | 9.94 |
| Te (day^{31 1 )} | 260 | 6.08 | 519 | 13.1 | 508 | 1.59 |
| s_pop | 1.41 | 0.279 | 1.82 | 0.631 | 2.42 | 0.794 |
| β_link | | | 0.0772 | 2.21 | 14.5 | 0.101 |
| Inter-individual variability | | | | | | |
| ω_M0 (%) | 50 | 4.67 | 49.9 | 4.67 | 50.3 | 4.7 |
| ω_KL (%) | 100 | 6.73 | 105 | 6.71 | 85.8 | 6.79 |
| ω_Kdi (%) | 111 | 10.3 | 78.8 | 11.4 | 83 | 11.8 |
| ω_Ri (%) | 184 | 13.7 | 175 | 12.3 | 176 | 14.4 |
| ω_KDpd (%) | 88.8 | 7.26 | 95.4 | 6.85 | 82.8 | 7.08 |
| ω_Rpd (%) | 80.8 | 8.14 | 88.9 | 8.29 | 105 | 8.51 |
| Residual variability | | | | | | |
| σ additive (g/L) | 0.369 | 7.25 | 0.353 | 7.03 | 0.344 | 7.92 |
| σ proportional (%) | 15.7 | 3.29 | 16.2 | 3.02 | 16.7 | 3.15 |

ALBN, baseline serum albumin normalized to the upper limit value;
B2MG, baseline β2-microglobulin;
M0, serum M-protein at baseline;
RSE, relative standard error.

Joint Modeling of Serum M-Protein and PFS

The joint model using the serum M-protein slope outperformed all models relying on serum M-protein in terms of Bayesian Information Criteria (BIC). with a 196-point decrease compared with the no-link model, that is, the parametric log-logistic model with no association between serum M-protein and PFS. The alternative models, based on current serum M-protein value or cumulative serum M-protein (area-under-serum M-protein), led to a BIC improvement <103. Comparison of joint models with different link functions is provided in Table F. In the best, final joint model, the longitudinal model still includes the same three covariates; however, only the presence of plasmacytomas remains on the PFS part. Parameter estimates obtained with the serum M-protein slope joint model are summarized in Table G. They were reasonably well estimated with low relative standard error for both fixed effects and variance components.

TABLE F
Parameter estimates of the PFS log-logistic model
without covariates and with significant covariates.
P-values of covariate effects were computed by Wald test.

| | Basic model | | Covariate model | | |
|---|---|---|---|---|---|
| | | RSE | | RSE | P-value |
| Fixed | Estimate | (%) | Estimate | (%) | (Wald-test) |
| Te (day⁻¹) | 260 | 8.56 | 283 | 9.2 | |
| S | 1.42 | 8.65 | 1.51 | 0.966 | |
| β1~PCYTOMA = | | | 0.737 | 34.7 | 0.00457 |
| Y | | | | | |
| β2~ALBN | | | −2.1 | 35.3 | 0.00281 |
| β3~MPROT | | | 0.472 | 33.5 | 0.00399 |

ALBN, baseline serum albumin normalized to the upper limit value;
MPROT, M-protein;
PCYTOMA, Y: presence of plasmacytomas;
PFS, progression-five survival;
RSE, relative standard error.

TABLE G
Parameter estimates values (relative standard
error %) of the best joint final model

| | Parameter | Relative standard | p-value |
|---|---|---|---|
| | estimates | error (%) | Wald test |
| Fixed effects | | | |
| Longitudinal submodel | | | |
| M0 (g/L) | 17 | 3.33 | |
| β1~ALBN | −1.41 | 15.8 | 2.34E−10 |
| β2~B2MG | 0.331 | 17 | 3.88E−09 |
| KL (day⁻¹) | 0.00627 | 8.4 | |
| β3~Non_IgG | 0.55 | 27.5 | 0.000277 |
| Kdi (L · mol−1 · day⁻¹) | 0.0138 | 8.73 | |
| Ri (day⁻¹) | 0.00579 | 14.6 | |
| KDpd (L · mol⁻¹ · | 0.188 | 7.36 | |
| day⁻¹) | | | |
| Rpd (day⁻¹) | 0.00952 | 10.6 | |
| Survival submodel | | | |
| Te (day⁻¹) | 459 | 11.9 | |
| S | 2.33 | 9.75 | |
| β4~PCYTOMA = Y | 0.858 | 36.3 | 0.00591 |
| β5~SlopeM | 11.9 | 7.66 | 4.46E−39 |
| Inter-individual variability | | | |
| standard deviation | | | |
| ω_M0 | 50.5 | 4.73 | |
| ω_KL | 88.7 | 6.76 | |
| ω_KDi | 61.9 | 9.7 | |
| ω_Ri | 105 | 9.76 | |
| ω_KDpd | 92 | 6.81 | |
| ω_Rpd | 110 | 9.33 | |
| Residual variability | | | |
| σ additive (g/L) | 0.411 | 7.23 | |
| σ proportional (%) | 15 | 3.65 | |

ALBN, baseline serum albumin normalized to the upper limit value;
B2MG, baseline β2-microglobulin;
M0, serum M-protein at baseline;
PCYTOMA, Y: presence to the upper of plasmacytomas;

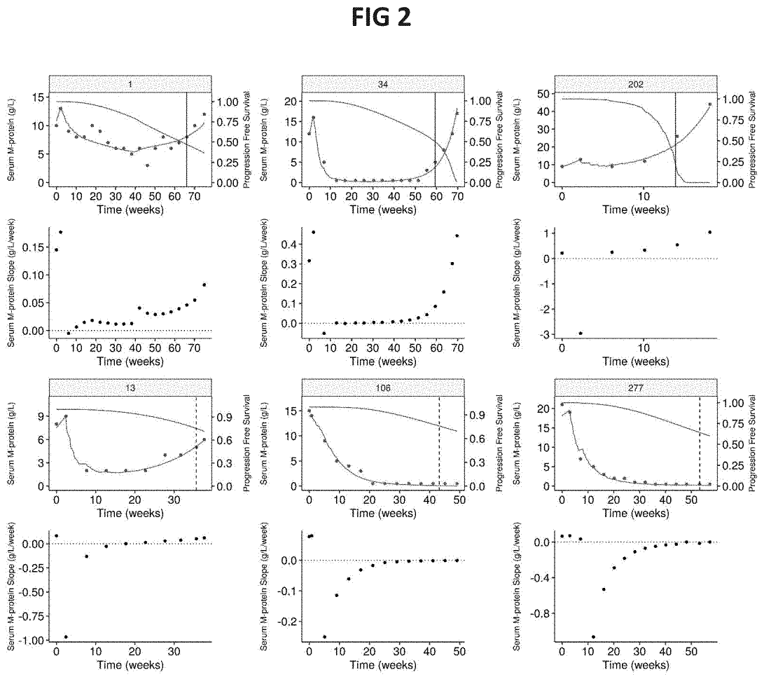

The estimated link between serum M-protein slope and PFS is high at 11.9, consistent with IMWG criteria, in which the decrease in serum M-protein in response to treatment is the main component directly impacting PFS. Thus, in case of an initial response, the serum M-protein decrease is associated with a current slope lower than 0 and hence a reduced risk of progression. The relationship among serum M-protein kinetics, slope, and PFS is illustrated in FIG. 2 for six representative patients who either had a PFS event or not. The PFS probability increased during tumor growth, i.e. when the serum M-protein slope increased. Furthermore, the baseline covariates were found to modify parameters of serum M-protein kinetics and PFS.

Model Evaluation

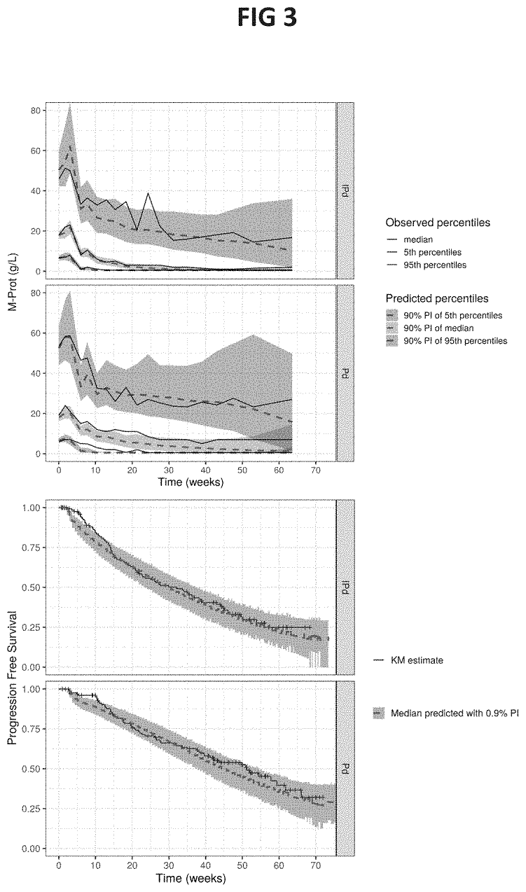

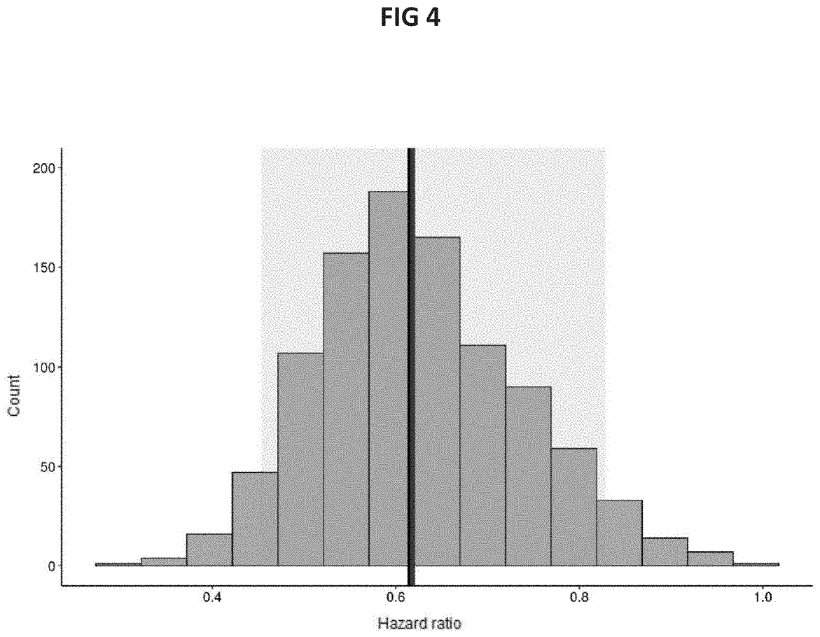

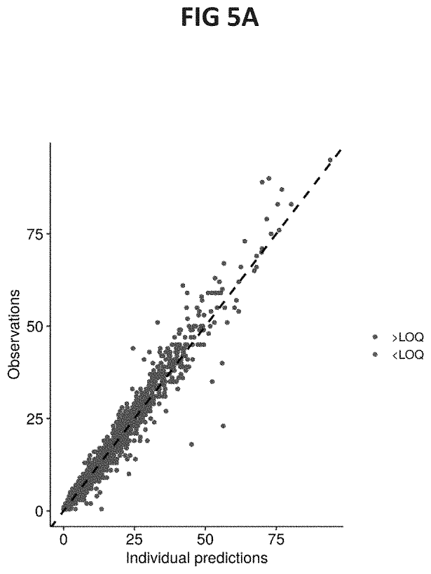

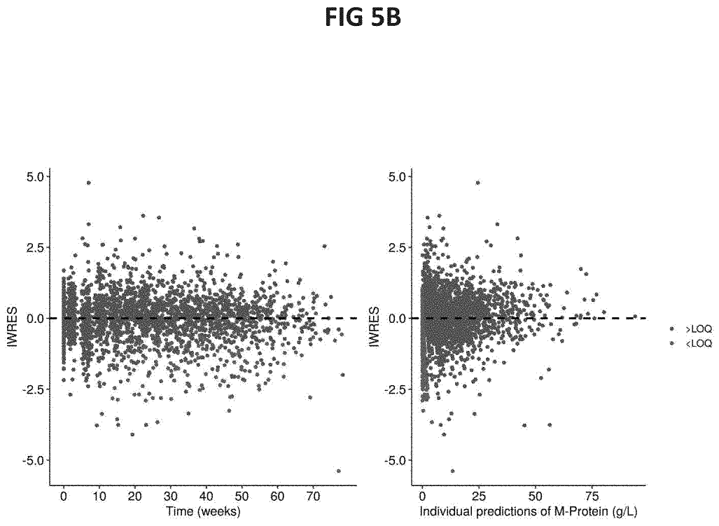

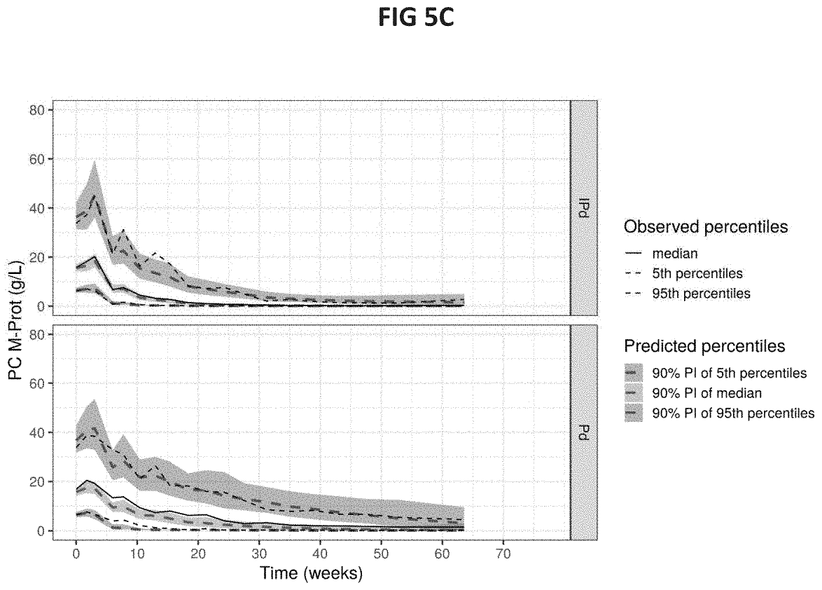

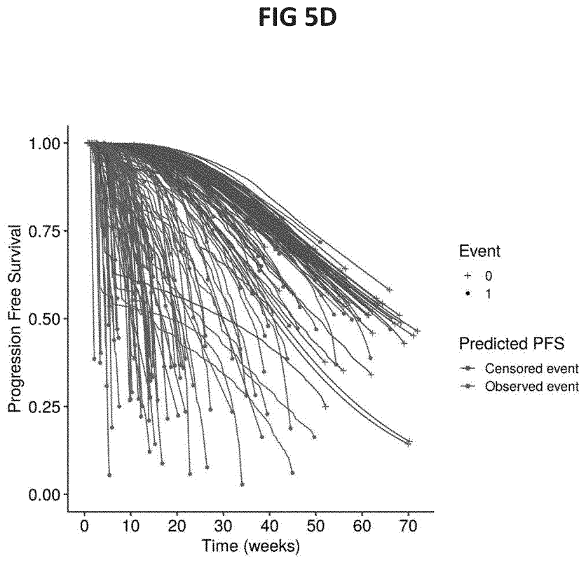

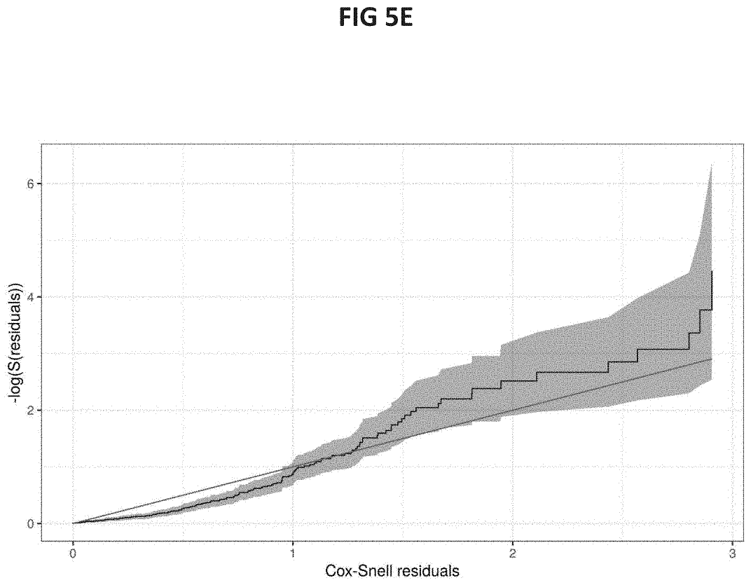

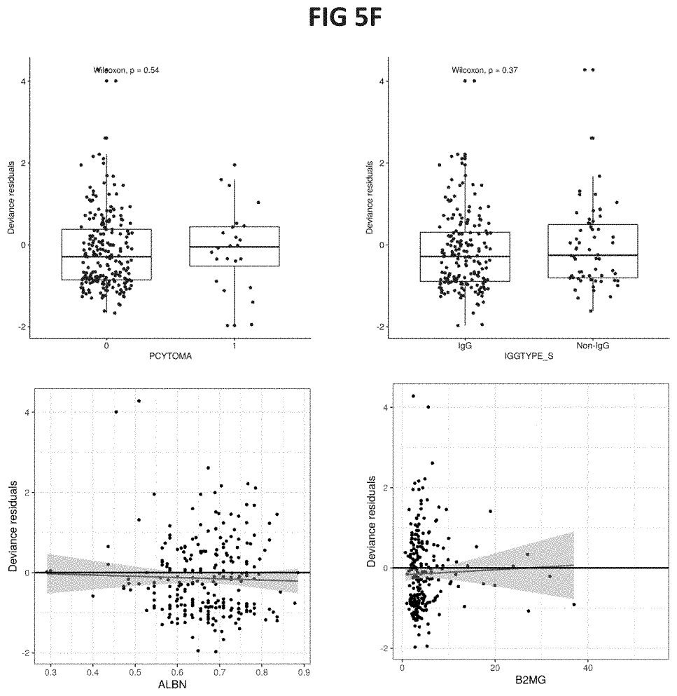

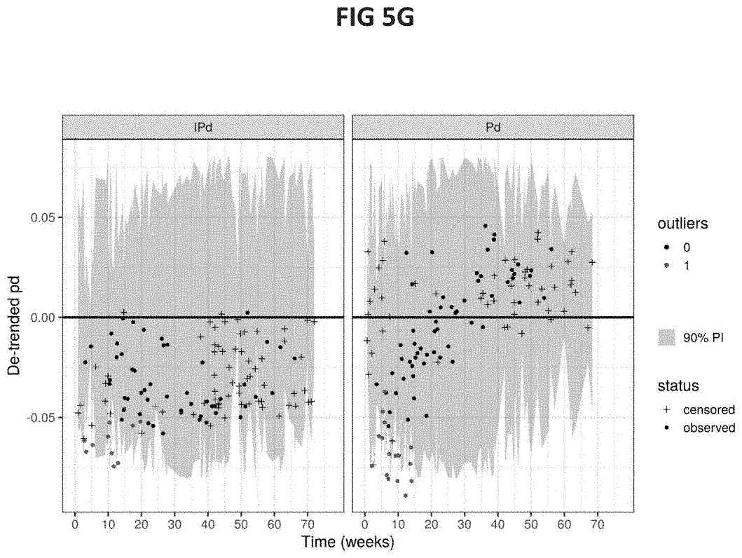

The various serum M-protein kinetic patterns could be well captured by the model, and predicted PFS probability is consistent with occurrent time of progression or censored event. FIG. 3 shows VPC plots generated for both longitudinal and PFS models by simulation of 1000 clinical trials under the final joint model, using the same design and patient characteristics as in the data. The model described reasonably well the observed serum M-protein and PFS data with observed median generally included in the 90%-prediction interval. However, unusual early event was observed, since the model did not capture a small group of patients who switched therapy without achieving PFS criteria. The final joint model also predicted well the HR observed between arms (FIG. 4), with observed HR close to the predicted median HR. Additional goodness-of-fit plots are presented in FIGS. 5A-5G.

Assessment of Covariates Effects

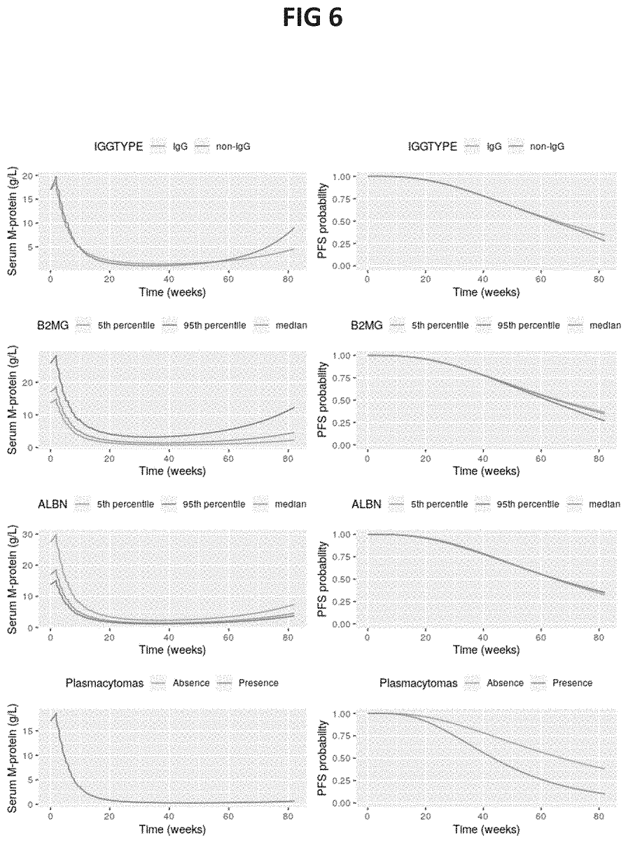

Simulations were performed to quantify the impact of each covariate using the population parameters and visualized in a typical patient (FIG. 6). The effect of covariates was assessed individually by setting others to their median value for continuous covariates and for the most frequent class for categorical covariates (i.e. IgG type). The effect of continuous covariates, baseline serum albumin and serum β2-microglobulin, were examined for variations within the 5^{th}-95^{th} database percentiles.

Non-IgG MM patients had similar behavior on serum M-protein kinetics for the first 60 weeks even with higher isatuximab exposure and tended to have more rapid tumor regrowth (i.e., re-increase in serum M-protein) afterwards compared to IgG MM patients. Similar PFS probability is predicted for non-IgG MM patients compared to IgG MM patients.

Patients with low baseline albumin and high β2-microglobulin levels are more likely to have higher baseline serum M-protein. However, the impact on the shape of M-protein profiles was a slightly faster tumor regrowth and slightly lower at end of treatment compared with other patients. Patients with plasmacytoma shared a similar PFS profile for 20 weeks, but tended to have lower PFS probability, up to 25% after 80 weeks.

Simulation of Monthly Dosing Regimen

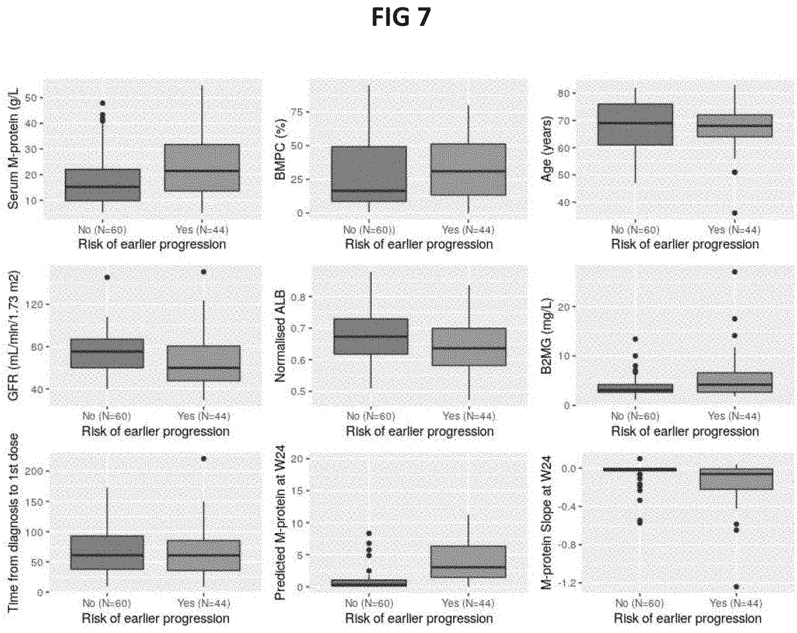

In the 1000 simulated trials, the median (min-max) number of patients at risk after 6 months (i.e., patients who had not progressed by 6 months) was 97 (86-107) in the Isa-Pd arm. In patients at risk at 6 months who switched to the isatuximab monthly dosing regimen after 6 months of QW-Q2W, median (5th-95th percentiles) progression was predicted to occur 2.29 (0.57-4.73) weeks earlier and HR predicted to be greater (0.7 vs 0.66) compared with the original Isa-Pd arm. Additionally, when considering the TTP criteria (i.e. increase in serum M-protein >25% with absolute increase >5 g/L), 44/104 (42.3%) patients who have not progressed at 6 months in the original Isa-Pd arm would have their serum M-protein regrow faster. Evaluation of baseline patient characteristics showed that these impacted patients have more disease burden at baseline, i.e. higher serum M-protein, higher bone marrow plasma cells (BMPC), and worse prognostic characteristics, such as longer time from diagnosis to first dose, lower eGFR, lower serum albumin, and higher serum β2-microglobulin with more frequent R-ISS II/III stage disease (80% vs 53.2%) (FIG. 7). Conversely, patients with no risk of earlier progression tend to have lower tumor burden (lower serum M-protein, lower BMPC) and better prognostic characteristics at baseline (higher eGFR and albumin, lower β2-microglobulin, more frequent R-ISS stage I). In addition, at 6 months they have significantly lower M-protein (median, 0.31 vs 3.04 g/L) and more stable response, with a serum M-protein slope close to 0 (i.e. M-protein level reached a plateau; median, −0.01 vs −0.06 g/L/day) and 85% of them have predicted response status of at least very good partial response.

### d) Discussion

A nonlinear joint model was developed in a MM setting, as joint models can provide efficient estimates and reduced bias of treatment effects on both time-to-event and longitudinal markers. We developed the TGI model using serum M-protein longitudinal data first and then the PFS model. Joint modeling was then performed to explore the best link between longitudinal serum M-protein and PFS. The model was built based on 256/307 patients from ICARIA-MM for whom serum M-protein data were used to evaluate treatment response.

Using longitudinal serum M-protein data from ICARIA-MM, we developed a TGI model from Claret et al. and compared it with the Wang et al. model, which was used for elotuzumab plus lenalidomide/dexamethasone (ELOQUENT-2) data [24,34]. We selected the Claret et al. model, which provided a better fit and included the combined pomalidomide/dexamethasone dose and isatuximab PK exposure as predictors. This allowed us to use this model to simulate serum M-protein response under other dosing regimens. This model accounts for three important clinical features of tumor progression in anticancer drug treatment, including the dynamics of tumor growth/production of serum M-protein, antitumor drug effect, and resistance to drug effect.

Furthermore, we studied the impact of numerous baseline covariates on serum M-protein kinetics and risk of progression. The significant baseline covariates in the joint model were Ig MM type, albumin, β2-microglobulin, and presence of plasmacytomas. Patients with low baseline albumin and high β2-microglobulin levels were more likely to have higher serum M-protein at baseline. Of note, these laboratory tests are part of the ISS and R-ISS staging systems, which are relevant for prognosis assessment, because patients with more advanced stage (i.e. ISS stage III) are less likely to respond to treatment. The presence of plasmacytomas likely induced lower PFS probability, consistent with results of exposure-response analyses [3]. In addition, the serum M-protein instantaneous slope was associated with PFS consistent with IMWG criteria, in which the serum M-protein decrease in response to treatment is the main component directly impacting PFS.

The simulation of typical patients indicated that non-IgG MM patients have similar behavior on serum M-protein kinetics for the first 60 weeks, even with higher steady-state isatuximab exposure, but tend to have more rapid tumor regrowth afterwards and similar PFS compared to IgG MM patients. The Ig MM type was also identified as the main contributor explaining isatuximab PK interindividual variability, with faster clearance in IgG MM patients. Elevated levels of IgG M-protein can lead to increased clearance of IgG-based monoclonal antibodies as a result of competition for the neonatal Fc receptor, which protects IgG from degradation [35-37]. This results in a two-fold lower exposure at steady-state in IgG MM compared with non-IgG MM patients.

However, in the exposure-response analyses, the Ig MM type was a predictor of ORR, but was no longer significant when C_{trough} at 4 weeks was included in the model. Additionally, Ig MM type was not a significant covariate in the univariate analysis for efficacy. Lastly, subgroup analyses showed that there was no significant difference in treatment effect of the Isa-Pd regimen over the Pd regimen on PFS or ORR for IgG versus non-IgG patients, with improved response rates with Isa-Pd versus Pd observed both for IgG and non-IgG patients [38]. Similar results were observed with daratumumab, as the linear clearance was ˜110% higher in IgG than non-IgG patients, leading to 70% higher predicted trough serum concentrations on day 1/cycle 3 in non-IgG patients [39]. Despite the difference in clearance levels between IgG and non-IgG patients, response rates in these populations were similar. The similar ORR is consistent with our finding, with the non-IgG type affecting the serum M-protein growth rate with similar behavior on serum M-protein kinetics for the first 60 weeks and faster regrowth thereafter. Therefore, the impact of Ig MM type (IgG vs non-IgG) on isatuximab exposure does not appear to be clinically meaningful.

The drug-disease modeling platform established based on ICARIA-MM data was further applied to predict the impact of using a hypothetical monthly dosing regimen after 6 months of isatuximab QW-Q2W in RRMM patients. In patients still on treatment, simulations of a hypothetical switch to monthly dosing after 6 months predicted progression to occur 2.3 weeks earlier compared with the original Isa-Pd arm, with 42.3% of patients having their serum M-protein regrow faster. Although we have limited patient numbers, impacted patients with earlier progression appeared to have more disease burden at baseline and worse prognostic characteristics. Patients with no risk of earlier progression tended to have lower tumor burden and better prognostic characteristics at baseline, with a stable, at least very good partial response at 6 months.

These results confirm the approved isatuximab QW-Q2W dosing regimen, which was selected for ICARIA-MM.

### e) Summary and Conclusion

Aim: Addition of isatuximab to pomalidomide/dexamethasone (Pd) significantly improved progression-free survival (PFS) in patients with relapsed/refractory multiple myeloma (RRMM). We aimed to characterize the relationship between serum M-protein kinetics and PFS in the Phase 3 ICARIA-MM trial, and to evaluate an alternative dosing regimen of isatuximab by simulation.

Methods: Data from the ICARIA-MM trial comparing isatuximab 10 mg/kg weekly for 4 weeks then every 2 weeks (QW-Q2W) in combination with Pd versus Pd in 256 evaluable RRMM patients were used. A joint model of serum M-protein dynamics and PFS was developed. Trial simulations were then performed to evaluate whether efficacy is maintained after switching to a monthly dosing regimen.

Results: The model identified instantaneous changes (slope) in serum M-protein as the best on-treatment predictor for PFS and baseline patient characteristics impacting serum M-protein kinetics (albumin and β2-microglobulin on baseline levels; non-IgG type on growth rate), and PFS (presence of plasmacytomas). Trial simulations demonstrated that switching to a monthly isatuximab regimen at 6 months would shorten median PFS by 2.3 weeks and induce 42.3% patients to progress earlier.

Conclusion: Trial simulations supported selection of the approved isatuximab 10 mg/kg QW-Q2W regimen and showed that switching to a monthly regimen after 6 months may reduce clinical benefit in the overall population. However, patients with good prognostic characteristics and a stable, very good partial response may switch to a monthly regimen after 6 months without compromising the risk of disease progression.

### (f) References for Examples 2 and 2A

1. Kumar S K, Rajkumar V, Kyle R A, et al. Multiple myeloma. Nat Rev Dis Primer. 2017; 3: 17046.
2. Thai H-T, Liu L, Koiwai K, et al. Exposure-response analysis and disease modeling for selection of optimal dosing regimen of isatuximab as single agent in patients with multiple myeloma. European Hematology Association Annual Meeting 2019: Abstract PF645.
3. Rachedi F, Koiwai K, Gaudel-Dedieu N, et al. Exposure-response analyses and disease modeling for selection and confirmation of optimal dosing regimen of isatuximab in combination treatment in patients with multiple myeloma. Blood. 2019; 134(Supplement 1):1897-1897.
4. Attal M, Richardson P G, Rajkumar S V, et al. Isatuximab plus pomalidomide and low-dose dexamethasone versus pomalidomide and low-dose dexamethasone in patients with relapsed and refractory multiple myeloma (ICARIA-MM): a randomised, multicentre, open-label, phase 3 study. The Lancet. 2019; 394(10214):2096-2107.
5. Moreau P, Dimopoulos M-A, Mikhael J, et al. Isatuximab, carfilzomib, and dexamethasone in relapsed multiple myeloma (IKEMA): a multicentre, open-label, randomised phase 3 trial. The Lancet 2021:397(10292):2361-2371.
6. SARCLISA® (isatuximab-irfc) injection, for intravenous use. Prescribing Information. March 2021. https://products.sanofi.us/Sarclisa/sarclisa.pdf. Accessed Jun. 16, 2021.
7. European Medicines Agency. Sarclisa, INN-Ixatuximab. Summary of product characteristics. 2021. https://www.ema.europa.eu/en/documents/product-information/sarclisa-epar-product-information_en.pdf. Accessed Jun. 16, 2021.
8. Ribba B, Holford N, Magni P, et al. A review of mixed-effects models of tumor growth and effects of anticancer drug treatment used in population analysis. CPT Pharmacomet Syst Pharmacol. 2014; 3(5):113.
9. Bruno R, Bottino D, de Alwis D P, et al. Progress and opportunities to advance clinical cancer therapeutics using tumor dynamic models. Clin Cancer Res. 2020; 26(8):1787-1795.
10. Desmée S, Mentré F, Veyrat-Follet C, Guedj J. Nonlinear mixed-effect models for prostate-specific antigen kinetics and link with survival in the context of metastatic prostate cancer: a comparison by simulation of two-stage and joint approaches. AAPS J. 2015; 17(3):691-699.
11. Tardivon C, Desmée S, Kerioui M, et al. Association between tumor size kinetics and survival in patients with urothelial carcinoma treated with atezolizumab: implication for patient follow-up. Clin Pharmacol Ther. 2019; 106(4):810-820.
12. Desmée S, Guedj J, Veyrat-Follet C, Comets E. Mechanistic joint modelling for longitudinal PSA and survival data in advanced metastatic prostate cancer. Annual Meeting of the Population Approach Group in Europe. 2017: Abstract 7154.
13. Thai H-T, Fau J, France M, Comets E, Veyrat-Follet C. Joint modeling of longitudinal tumor burden and time-to-event data to predict survival: application to aflibercept in second line metastatic colorectal cancer. Annual Meeting of the Population Approach Group in Europe. 2015: Abstract 3527.
14. Durie B G M, Harousseau J-L, Miguel J S, et al. International uniform response criteria for multiple myeloma. Leukemia. 2006; 20(9):1467-1473.
15. Chanu P, Claret L, Bruno R, Carpenter S, Woolridge J, Cronier D. PK/PD relationship of the monoclonal anti-BAFF antibody tabalumab in combination with bortezomib in patients with previously treated multiple myeloma: comparison of serum M-protein and serum free light chains as predictors of progression free survival. Annual Meeting of the Population Approach Group in Europe. 2013: Abstract 2732.
16. Marchand M, Claret L, Losic N, Puchalski T, Bruno R. Population pharmacokinetics and exposure-response analyses to support dose selection of daratumumab in multiple myeloma patients. Annual Meeting of the Population Approach Group in Europe. 2013: Abstract 2668.
17. Bruno R, Jonsson F, Zaki M, et al. Simulation of clinical outcome for pomalidomide plus low-dose dexamethasone in patients with refractory multiple myeloma based on week 8 M-protein response. Blood 2011; 118(21):1881-1881.
18. Jonsson F, Ou Y, Claret L, et al. A Tumor growth inhibition model based on M-protein levels in subjects with relapsed/refractory multiple myeloma following single-agent carfilzomib use. CPT Pharmacomet Syst Pharmacol. 2015; 4(12):711-719.
19. Thai H-T, Liu L, Semiond D, et al. Model-based drug development to support isatuximab dosing regimen selection in Phase II multiple myeloma patients. Annual Meeting of the Population Approach Group in Europe. 2016: Abstract 6018.
20. Fau J, El-Cheikh R, Brillac C, et al. Drug-disease interaction and time-dependent population pharmacokinetics of isatuximab in relapsed/refractory multiple myeloma patients. CPT Pharmacomet Syst Pharmacol. 2020; 9(11):649-658.
21. Jacqmin P, Snoeck E, Schaick E A van, et al. Modelling response time profiles in the absence of drug concentrations: definition and performance evaluation of the K-PD model. J Pharmacokinet Pharmacodyn. 2007; 34(1):57-85.
22. Li Y, Xu Y, Liu L, Wang X, Palmisano M, Zhou S. Population pharmacokinetics of pomalidomide. J Clin Pharmacol. 2015; 55(5):563-572.
23. Spoorenberg S M C, Deneer V H M, Grutters J C, et al. Pharmacokinetics of oral vs. intravenous dexamethasone in patients hospitalized with community-acquired pneumonia. Br J Clin Pharmacol. 2014; 78(1):78-83.
24. Claret L, Girard P, Hoff P M, et al. Model-based prediction of phase III overall survival in colorectal cancer on the basis of phase II tumor dynamics. J Clin Oncol. 2009; 27(25):4103-4108.
25. Chanu P, Claret L, Marchand M, Losic N, Puchalski T, Bruno R. Population pharmacokinetic/pharmacodynamic models to support dose selection of daratumumab in multiple myeloma patients. Annual Meeting of the Population Approach Group in Europe. 2014: Abstract 3281.
26. Jonsson F, Claret L, Knight R, et al. A longitudinal tumor growth inhibition model based on serum M-protein levels in patients with multiple myeloma treated by dexamethasone. Annual Meeting of the Population Approach Group in Europe. 2010: Abstract 1705.
27. Richardson P G, Siegel D S, Vij R, et al. Pomalidomide alone or in combination with low-dose dexamethasone in relapsed and refractory multiple myeloma: a randomized phase 2 study. Blood. 2014; 123(12):1826-1832.
28. Ayral G, Si Abdallah J, Magnard C, Chauvin J. A novel method based on unbiased correlations tests for covariate selection in nonlinear mixed effects models: The COSSAC approach. CPT Pharmacomet Syst Pharmacol. 2021; 10(4):318-329.
29. Lavielle M, Ribba B. Enhanced method for diagnosing pharmacometric models: random sampling from conditional distributions. Pharm Res. 2016; 33(12):2979-2988.
30. Traynard P, Ayral G, Twarogowska M, Chauvin J. Efficient pharmacokinetic modeling workflow with the MonolixSuite: A case study of remifentanil. CPT Pharmacomet Syst Pharmacol. 2020; 9(4):198-210.
31. Therneau T M, Grambsch P M, Fleming T R. Martingale-based residuals for survival models. Biometrika. 1990; 77(1):147-160.
32. Cerou M, Lavielle M, Brendel K, Chenel M, Comets E. Development and performance of npde for the evaluation of time-to-event models. Pharm Res. 2018; 35(2).
33. Friberg L, de Greef R, Kerbusch T, Karlsson M. Modeling and simulation of the time course of asenapine exposure response and dropout patterns in acute schizophrenia. Clin Pharmacol Ther. 2009; 86(1):84-91.
34. Wang Y, Sung C, Dartois C, et al. Elucidation of relationship between tumor size and survival in non-small-cell lung cancer patients can aid early decision making in clinical drug development. Clin Pharmacol Ther. 2009; 86(2): 167-174.
35. Ryman J T, Meibohm B. Pharmacokinetics of monoclonal antibodies. CPT Pharmacomet Syst Pharmacol. 2017; 6(9):576-588.
36. Xu X S, Schecter J M, Jansson R, Yan X. Response to “The role of FcRn in the pharmacokinetics of biologics in patients with multiple myeloma.” Clin Pharmacol Ther. 2017; 102(6):905.
37. Jacobs J, Mould D. The role of FcRn in the pharmacokinetics of biologics in patients with multiple myeloma. Clin Pharmacol Ther. 2017; 102(6):903-904.
38. Food and Drug Administration Center For Drug Evaluation And Research. BLA Multi-disciplinary Review and Evaluation BLA 761113 Sarclisa (isatuximab). 2016. https://www.accessdata.fda.gov/drugsatfda_docs/nda/2020/761113Orig1s000MultidisciplineR.pdf. Accessed Jun. 16, 2021.
39. Yan X, Clemens P L, Puchalski T, et al. Influence of disease and patient characteristics on daratumumab exposure and clinical outcomes in relapsed or refractory multiple myeloma. Clin Pharmacokinet. 2018; 57(4):529-538.

### Example 2A: Supplementary Information for Example 2

Pharmacokinetic Model for Isatuximab

The equations of the structural pharmacokinetic model for isatuximab were as follows:

$$\frac{d {A}_{i c}}{d t} = - {k}_{12} * {A}_{i c} + {k}_{21} {A}_{i p} - \frac{C L l i n ( t )}{{V}_{i c}} * {A}_{i c} - \frac{{V}_{m}}{{K}_{m} + {C}_{i c}} * {A}_{i c} + l n ( t )$$

$$\frac{d {A}_{i p}}{d t} = {k}_{12} * {A}_{i c} - {k}_{21} {A}_{i p}$$

$$CLlin = CLinf * exp ( C L m * ( 1 - \frac{t i m {e}^{γ}}{K C {L}^{γ} + t i m {e}^{γ}} ) )$$

where A_{ic} C_{ic} and V_{ic} are the amount, concentration, and volume of distribution of isatuximab in the central compartment, Aᵢₚ and Vᵢₚ are the amount and volume of distribution of isatuximab in the peripheral compartment, k₁₂ and k₂₁ are the first-order rate constants between central and peripheral compartment, Vₘ and Kₘ are Michaelis-Menten parameters, with Kₘ representing the drug concentration value at which the elimination rate is half the maximum (Vₘ), and In(t) is the infusion rate. In the time-varying function for linear clearance (CLlin), CL_{inf} is the linear CL at steady-state, CLₘ is the maximum change in CL over time, KCL is the time at which clearance is reduced by half of the maximal reduction; and γ, the shape parameter describing the sigmoidicity degree.

K-PD Model for Pomalidomide and Dexamethasone

The kinetics of combined pomalidomide (p) and dexamethasone (d) was described by a simple virtual one compartment with bolus input as presented in detail below:

$$\frac{d {A}_{p}}{d t} = - K D {E}_{p} * {A}_{p} \\ \frac{d {A}_{d}}{d t} = - K D {E}_{d} * {A}_{d}$$

where KDEₚ and KDE_{d} represent the elimination rate constant for pomalidomide and dexamethasone, respectively.

Mixtran Code of the Joint Model

Model description: Joint model for M-protein and progression-free survival (PFS)

PK for Isa, K-PD for Pom Dex and parameters: M0,KL,KDi,Ri,KDpd,Rpd,Te,s,E,beta

```
[LONGITUDINAL]
input = 1Ri,KDpd,Rpd,Te,s,E,beta,CLinf,CLm,KCL,gamma,V,Q,V2,Vm,Km,Tstart
CLinf={use=regressor}
CLm={use=regressor}
KCL={use=regressor}
gamma= {use=regressor}
V={use=regressor}
Q={use=regressor}
V2={use=regressor}
Vm={use=regressor}
Km={use=regressor}
Tstart={use=regressor}
PK:
compartment(cmt=1 , amount=Aic,volume=V)
iv(cmt=1,adm=1)
comp artment(cmt=2, amount=Aip,volume=V2)
comp artment(cmt=3 ,amount=Ap)
iv(adm=3 ,cmt=3)
comp artment(cmt=4, amount=Ad)
iv(adm=4,cmt=4)
EQUATION:
odeType=stiff
;Initial conditions
t_0 =0
Aic_0 =0
Aip_0 =0
Ap_0 =0
Ad_0 =0
M_0 =MO
k12 =QN
k21 =QN2
Cic =AicN
ddt Aic = − 1/V*CLinf*( exp( CLm*(1 − 1/((KCL/t)Agamma + 1) ) ) )*Aic − Vm*Aic/( Km+Cic ) −
k12*Aic + k21*Aip
ddt_Aip = k12*Aic− k21*Aip
KDEp=3.51
KDEd=2.42
ddt Ap=−KDEp*Ap
ddt Ad=−KDEd*Ad
MW_ISA=150000
MW_POM=273.24
MW_DEX=392.46
Cp=Ap/(58.3*0.73);V/F=58.3 F=0.73
Cd=Ad/76.3
CiM=Cic/MW_ISA*1000 ;M
CpM=Cp/MW_POM*1000 ;M
CdM=Cd/MW_DEX*1000 ;M
if M<150
dM = KL*M − KDi*exp(−Ri*t)*CiM*M − KDpd*exp(−Rpd*t)*(CpM+CdM)*M
else
dM = 0
end
ddt_M = dM
SlopeM=dM
if t < Tstart
hoz = 0
else
hoz = s/Te * (t/Te) (s−1) / (1+(t/Te) s)*E*exp(beta*SlopeM)
end
ddt_H=haz
S=exp(−H)
DEFINITION:
PFS = {type=event,
  maxEventNumber=1,
  rightCensoringTime=540,
  hazard=haz}
OUTPUT:
output={M,PFS}
```

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

1. A method of treating a human individual having multiple myeloma, comprising:
administering isatuximab to the individual at a weekly dose of 10 mg/kg of a first one-month cycle;
administering the isatuximab at a dose of 10 mg/kg once every two weeks of a one-month cycle for at least 11 cycles following the first one-month cycle; and
administering the isatuximab at a monthly dose of 10 mg/kg for one or more additional one-month cycles following the at least 11 cycles.

2. A method of treating a human individual having multiple myeloma, comprising:
administering an anti-CD38 antibody to the individual at a weekly dose of 10 mg/kg of a first one-month cycle;
administering the anti-CD38 antibody at a dose of 10 mg/kg once every two weeks of one or more one-month cycles after the first one-month cycle until the individual achieves a response of at least very good partial response (VGPR); and
administering the anti-CD38 antibody at a dose of 10 mg/kg once a month for one or more additional one-month cycles after the individual achieves the response of at least VGPR.

3. A method of treating human individuals having multiple myeloma, comprising:
administering an anti-CD38 antibody to the individuals at weekly dose of 10 mg/kg of a first one-month cycle;
administering the anti-CD38 antibody at a dose of 10 mg/kg on once every two weeks of one or more one-month cycles after the first one-month cycle;
measuring the individuals' responses to the treatment at one or more time points during the one or more one-month cycles after the first one-month cycle and selecting the individuals who have at least a Very Good Partial Response (VGPR); and
administering the anti-CD38 antibody at a dose of 10 mg/kg once a month for one or more additional one-month cycles to the selected individuals.

4. A method of treating a human individual having multiple myeloma, comprising:
measuring the individual's serum and urine M-protein at a first time point prior to administration of an anti-CD38 antibody;
administering the anti-CD38 antibody to the individual at a weekly dose of 10 mg/kg of a first one-month cycle;
administering the anti-CD38 antibody at a dose of 10 mg/kg once every two weeks of one or more one-month cycles after the first one-month cycle;
measuring the individual's serum and/or urine M-protein at a second time point during the at least one or more one-month cycles after the first one-month cycle, and
administering the anti-CD38 antibody at a monthly dose of 10 mg/kg for one or more additional one-month cycles if (a) the individual's serum M-protein level at the second time point is reduced by at least 90% as compared to the individual's serum M-protein level at the first time point and (b) the individual's urine M-protein level at the second time point is less than 100 mg/24 hours.

5. A method of treating a human individual having multiple myeloma, comprising:
measuring the individual's serum and/or urine M-protein level prior to administration of anti-CD38 antibody;
administering the anti-CD38 antibody to the individual at a weekly dose of 10 mg/kg of a first one-month cycle;
administering the isatuximab at a dose of 10 mg/kg once every two weeks of one or more one-month cycles after the first one-month cycle until (a) the individual's serum M-protein level at reduced by at least 90% as compared to the serum M-protein level prior to the administration of the anti-CD38 antibody and (b) the individual's urine M-protein level is less than 100 mg/24 hours; and
administering the anti-CD38 antibody at a dose of 10 mg/kg once a month for one or more additional one-month cycles after (a) the individual's serum M-protein level is determined to be reduced by at least 90% as compared to the individual's serum M-protein level at the first time point and (b) the individual's urine M-protein level is determined to be less than 100 mg/24 hours.

6. The method of claim 5, wherein (a) the reduction in the individual's serum M-protein level and (b) the individual's urine M-protein level of less than 100 mg/24 hours are maintained for at least about 12 months prior to administering the anti-CD38 antibody at a dose of 10 mg/kg once every 28-day cycle.

7. The method of claim 2 or 3, wherein the response to treatment is measured by assessing M-protein level in the blood and/or urine of the individual or individuals.

8. The method of any one of claims 5, 6, and 7, wherein the M-protein level in the blood and/or urine is assessed via immunofixation and/or electrophoresis.

9. The method of any one of claims 2, 3, 7, and 8, wherein the response of at least VGPR is maintained for at least about 6 months prior to administering the anti-CD38 antibody once a month for one or more one-month cycles.

10. The method of claim 9, wherein the response of at least VGPR is maintained for at least about 12 months prior to administering the anti-CD38 antibody once a month for one or more one-month cycles.

11. The method of any one of claims 2-10, wherein the anti-CD38 antibody is administered at a dose of 10 mg/kg once every two weeks of the one or more one-month cycles for at least 11 cycles prior to administering the anti-CD38 antibody once a month for one or more additional one-month cycles.

12. The method of any one of claims 1-11, wherein the anti-CD38 antibody is administered at a dose of 10 mg/kg once every other week of one or more one-month cycles for at least 23 cycles prior to administering the anti-CD38 antibody once a month for one or more additional one-month cycles

13. The method of any one of claims 1-12, wherein the treatment extends the progression free survival (PFS) of the individual.

14. The method of any one of claims 2-4 and 9-13 wherein the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (VL) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6).

15. The method of any one of claims 2-4 and 9-14, wherein the anti-CD38 antibody comprises a heavy chain variable region (VH) comprising an amino acid sequence of SEQ ID NO: 7 and a light chain variable region (VL) comprising an amino acid sequence of SEQ ID NO: 8 or SEQ ID NO: 9.

16. The method of any one of claims 2-4 and 9-15, wherein the anti-CD38 antibody is isatuximab.
